SEQUENCE LISTING (2 sequences)

SEQ ID NO: 1 - DNA, 21 bp, Artificial Sequence
  PCR PRIMER
gggtttgttcctatctctttg

SEQ ID NO: 2 - DNA, 19 bp, Artificial Sequence
  PCR PRIMER
gacctggcgagcccacgag

(19) United States
(12) Patent Application Publication
(10) Pub. No.: US 2013/0053278 A1
(43) Pub. Date: Feb. 28, 2013

(54) TOTIPOTENT, NEARLY TOTIPOTENT OR PLURIPOTENT MAMMALIAN CELLS HOMOZYGOUS OR HEMIZYGOUS FOR ONE OR MORE HISTOCOMPATIBILITY ANTIGENS GENES

(71) Applicants: Michael D. WEST, Mill Valley, CA (US); R. Geoffrey Sargent, San Lorenzo, CA (US)

(72) Inventors: Michael D. WEST, Mill Valley, CA (US); R. Geoffrey Sargent, San Lorenzo, CA (US)

(21) Appl. No.: 13/585,380

(22) Filed: Aug. 14, 2012

Related U.S. Application Data
(62) Division of application No. 12/083,799, filed on Apr. 13, 2009, now abandoned.
(60) Provisional application No. 60/729,173, filed on Oct. 20, 2005.

(51) Int. Cl. C40B 40/02; C12N 5/10; C12N 15/85; C12N 15/01

(52) U.S. Cl. 506/14; 435/455; 435/325

(57) ABSTRACT

The present invention relates to totipotent, nearly totipotent and pluripotent stem cells that are hemizygous or homozygous for MHC antigens and methods of making and using them. These cells are useful for reduced immunogenicity during transplantation and cell therapy. The cells of the present invention may be assembled into a bank with reduced complexity in the MHC genes.

### RELATED APPLICATION DISCLOSURE

This application is a Divisional of U.S. Ser. No. 12/083,799, filed Apr. 18, 2008, pending, which is a U.S. National Stage of International Application No. PCT/US2006/040985, filed Oct. 20, 2006, which claims the benefit of U.S. Provisional Application No. 60/729,173 filed Oct. 20, 2005, each of which is hereby incorporated by reference in its entirety.

The sequence listing in the file named “75820o004012.txt” having a size of 808 bytes that was created Aug. 14, 2012 is hereby incorporated by reference in its entirety.

### BACKGROUND OF THE INVENTION

Advances in stem cell technology, such as the isolation and use of human embryonic stem cells (“hES” cells), constitute an important new area of medical research. hES cells have a demonstrated potential to differentiate into any and all of the cell types in the human body, including complex tissues. This has led to the suggestion that many diseases resulting from the dysfunction of cells may be amenable to treatment by the administration of hES-derived cells of various differentiated types (Thomson et al., Science 282:1148-7, (1998)). Nuclear transfer studies have demonstrated that it is possible to transform a somatic differentiated cell back to a totipotent state such as that of embryonic stem cells (“ES”) or embryonic derived cells (“ED”) (Cibelli et al., Nature Biotech 16:642-646, (1998)). The development of technologies to reprogram somatic cells back to a totipotent ES cell state such as by the transfer of the genome of the somatic cell to an enucleated oocyte and the subsequent culture of the reconstructed embryo to yield ES cells, often referred to as somatic cell nuclear transfer (SCNT), offers a means to deliver ES-derived somatic cells with a nuclear genotype of the patient (Lanza et al., Nature Medicine 5:975-977, (1999)). It is expected that such cells and tissues would not be rejected, despite the presence of allogeneic mitochondria (Lanza et al., Nature Biotech 20:689-696, (2002)). Nevertheless, there remains a need for improvements in methods to supply cells and tissues that will not be rejected by a patient, especially where there is not sufficient time to perform SCNT either because the medical condition is acute and transplantation is needed acutely, or because considerable genetic modification of the cells is preferred and the patient's health does not permit enough time for the modification.

### 1. Histocompatibility and Transplant Rejection

Histocompatibility is a largely unsolved problem in transplant medicine. Rejected transplanted tissue is rejected as a result of an adaptive immune response to alloantigens on the grafted tissue by the transplant recipient. The alloantigens are “non-self” proteins, i.e., antigenic proteins that vary among individuals in the population and are identified as foreign by the immune system of a transplant recipient. The antigens on the surfaces of transplanted tissue that most strongly evoke rejection are the blood group (ABC)) antigens, the major histocompatibility complex (MHC) proteins and, in the case of humans, the human leukocyte antigen (HLA) proteins. Any and all of these antigens are referred to herein as Histocompatibility antigens.

The blood group antigens were first described by Landsteiner in 1900. Compatibility of the blood group antigens of the ABO system of a vascularized organ or tissue transplant with those of the transplant recipient is generally required. But blood group compatibility may be unnecessary for many types of cell transplants that lack vascular endothelium.

The HLA proteins are encoded by clusters of genes that form a region located on human chromosome 66 known as the Major Histocompatibility Complex, or MHC, in recognition of the important role of the proteins encoded by the MHC loci in graft rejection. Accordingly, the HLA proteins are also referred to as MHC proteins. The MHC genes and proteins will be used interchangeably in this application as the application encompasses human and non-human animal applications. Class I MHC proteins are found on virtually all of the nucleated cells of the body. The class I MHC proteins bind peptides present in the cytosol and form peptide-MHC protein complexes that are presented at the cell surface, where they are recognized by cytotoxic CD8+ T cells. Class II MHC proteins are usually found only on antigen-presenting cells such as B lymphocytes, macrophages, and dendritic cells. The class II MHC proteins bind peptides present in a cell's vesicular system and form peptide-MHC protein complexes that are presented at the cell surface, where they are recognized by CD4+ T cells.

Unfortunately for those in need of transplants, the frequency of T cells in the body that are specific for non-self MHC molecules is relatively high, with the result that differences at MHC loci are the most potent critical elicitors of rejection of initial grafts. Rejection of most transplanted tissues is triggered predominantly by the recognition of class I MHC proteins as non-self proteins. T cell recognition of foreign antigens on the transplanted tissue sets in motion a chain of signaling and regulatory events that causes the activation and recruitment of additional T cells and other cytotoxic cells, and culminates in the destruction of the transplanted tissue. (Charles A. Janeway et al., Immunobiology, Garland Publishing, Mew York, N.Y., 2001, p. 524).

### 2. The Genes Encoding MHC Proteins

The MHC genes are polygenic: each individual possesses multiple, different. MHC class I and MHC class II genes. The MHC genes are also polymorphic: many variants of each gene are present in the human and non-human population. In fact, the MHC genes are the most polymorphic genes known. Each MHC Class I receptor consists of a variable alpha chain and a relatively conserved beta2-microglobulin chain. Inactivation of beta2-microglobulin by genetic modification may reduce or eliminate the expression of functional class I MHC antigens (see, for example, U.S. Pat. Nos. 5,514,752; 6,139,835; 5,670,148; and 5,413,923). The resulting cells may be useful as universal donor cells, though they would be expected to have an impaired ability to present antigens that may pose a health risk to the organism. Three different, highly polymorphic class alpha chain genes have been identified: HLA-A, HLA-B, and HLA-C. Variations in the alpha chain account for all of the different class I MHC genes in the population. MHC Class II receptors are also made up of two polypeptide chains, an alpha chain and a beta chain, both of which are polymorphic. In humans, there are three pairs of MHC class II alpha and beta chain genes, called HL-DR, HLA-DP, and HLA-DQ. Frequently, the HLA-DR cluster contains an extra gene encoding a beta chain that can combine with the DR alpha chain. Thus, an individuals three MHC Class II genes can give rise to four different MHC Class II molecules.

In humans, the genes encoding the MHC class alpha chains and the MHC class II alpha and beta chains are clustered on the short arm of chromosome 6 in a region that extends from 4 to 7 million base pairs that is called the major Histocompatibility complex. Every person usually inherits a copy of each HLA gene from each parent. If an individuals two alleles for a particular MHC locus encode structurally different proteins, the individual is heterozygous for that MHC allele. If an individual has two MHC alleles that encode the same MHC molecule, the individual is homozygous for that MHC allele. Because there are so many different variants of the MHC alleles in the population, most people have heterozygous MHC alleles.

### 3. Matching MHC Types to Inhibit Rejection of Transplants

Since the recognition that non-self MHC molecules are a major determinant of graft rejection, much effort has been put into developing assays to identify the NBC types present on the cells of tissue to be transplanted and on the cells of transplant recipients, so that the type of MHC molecules on the transplant tissue can be matched with those of the recipient. The detection of MHC antigens, or tissue typing, is performed by various means.

At present, tissue typing to match the HLA antigens of transplant tissue with those of a recipient is usually limited to the Class I HLA-A and -B antigens, and the Class II HLA-DR antigens. Most transplant donors are unrelated to the transplant recipient. Finding a tissue type to match that of the recipient usually involves matching the blood type and as many as possible of the 6 HLA alleles—two for each of the HLA-A, -B, and -DR locus. Transplant centers do not usually consider potential incompatibilities at other HLA loci, such as HLA-C and HLA-DPB1, though mismatches at these loci can also contribute to rejection. Considering only the combinations of maternal and paternal alleles of the HLA-A, HLA-B, and HLA-DR loci identified to date, there is a complexity of billions of possible tissue types. The task of matching HLA types of organs for transplant is simplified in that HLA-A and HLA-B are usually identified serologically. The number of HLA antigens identified serologically is considerably less than the number of different HLA antigens based on DNA sequencing. The World Health Organization (WHO) has recognized 28 distinct antigens in the HLA-A locus and 59 in the HLA-B locus, based on serological typing. Matching organs is also simplified to some extent by the fact that some alleles are much more common than others.

The frequencies with which the various alleles appear in a population is not random. It depends on the racial makeup of the population. Dr. Motomi Mori has determined the frequencies at which thousands of different haplotypes of HLA-A, -B, and -DR loci appear in Caucasian, African-American, Asian-American, and Native American populations. Each haplotype is a particular combination of HLA-A, HLA-B, and HLA-DR loci that is present on a single copy of chromosome no. 6. In interpreting haplotype frequency data, one must bear in mind that cells of patients and organs are diploid and have an HLA type that is the product of the HLA haplotypes of the chromosomes inherited from both parents.

### 4. Rejection Triggered by Minor Histocompatibility Antigens

Matching the MEC molecules of a transplant to those of the recipient significantly improves the success rate of clinical transplantation. But it does not prevent rejection, even when the transplant is between HLA-identical siblings. This is so because rejection is also triggered by differences between the minor Histocompatibility antigens. These polymorphic antigens are actually “non-self” peptides bound to MHC molecules on the cells of the transplant tissue. The rejection response evoked by a single minor Histocompatibility antigen is much weaker than that evoked by differences in MHC antigens, because the frequency of the responding T cells is much lower (Janeway et al., supra, page 525). Nonetheless, differences between minor Histocompatibility antigens will often cause the immune system of a transplant recipient to eventually reject a transplant, even where there is a match between the MHC antigens, unless immunosuppressive drugs are used.

### 5. Inadequate Supply of Cells, Tissues, and Organs for Transplant

The number of people in need of cell, tissue, and organ transplants is far greater than the available supply of cells, tissues, and organs suitable for transplantation. Under these circumstances, it is not surprising that obtaining a good match between the MHC proteins of a recipient and those of the transplant is frequently impossible, and many transplant recipients must wait for an MHC-matched transplant to become available, or accept a transplant that is not MHC-matched. If the latter is necessary, the transplant recipient must rely on heavier doses of immunosuppressive drugs and face a greater risk of rejection than would be the case if MHC matching had been possible. There is presently a great need for new sources of cells, tissues, and organs suitable for transplantation that are histocompatible with the patients in need of such transplants.

### SUMMARY OF THE INVENTION

The present invention provides totipotent, nearly totipotent and pluripotent stem cells that are hemizygous or homozygous for MHC antigens and methods of making and using them. These cells are useful for reduced immunogenicity during transplantation and cell therapy. The cells of the present invention may be assembled into a bank with reduced complexity in the MHC genes.

In one embodiment, the invention provides a totipotent, nearly totipotent or pluripotent stem cell that is hemizygous or homozygous for at least one MHC allele present in a human or non-human animal population. The cells of the invention may be any blood group and generated from a male or female. In preferred embodiments, the cells are O-negative and generated from a female. Gene targeting and/or loss of heterozygosity may be used to generate the hemizygous or homozygous MHC allele. In a specific embodiment, the invention provides In a specific embodiment, the invention provides a stem cell that is homozygous for at least one MHC allele present in a human or non-human animal population. Stem cells that are homozygous for at least one MHC allele may be generated by gene targeting to arrive at a hemizygous allele and then by loss of heterozygosity to arrive at a homozygous allele. The cells of the invention may further comprise one or more drug selectable markers. Drug selectable markers may be used to positively or negatively select cells that are hemizygous homozygous for at least one MHC allele

In certain embodiment, the cells of the invention also comprise nucleic acid sequences that encode recognition sequences for recombinases such as Ore/LoxP or FLP/FRT, and/or recognition sequences encoding endonucleases such as I-SceI.

In another embodiment, the invention provides a totipotent, nearly totipotent or pluripotent stem cell that is nullizygous for one or more (preferably all) MHC alleles present in a human or non-human animal population, wherein gene targeting and/or loss of heterozygosity is used to generate the cell that is nullizygous for all MHC alleles.

In another embodiment, the invention provides a bank of totipotent, nearly totipotent and/or pluripotent stem cells, comprising a library of human or non-human animal stem cells, each of which cells is hemizygous or homozygous for at least one MHC allele present in a human or non-human animal population The bank of stem cells may comprise stem cells that are hemizygous or homozygous for different sets of MHC alleles relative to the other members in the bank of stem cells. Gene targeting and/or loss of heterozygosity may be used to generate the hemizygous or homozygous MHC alleles.

In another embodiment, the invention provides a method of generating a stem cell hemizygous for at least one MHC allele, comprising deleting one of the two MHC alleles in a stem cell by gene targeting. In another embodiment, the invention provides a method of generating a stem cell homozygous for at least one MHC allele, comprising providing a stem cell that is hemizygous for at least one MHC allele and using loss of heterozygosity to generate a stem cell homozygous for at least one MHC allele. The methods of the invention may further comprise destabilizing or inactivating p53 by expressing the human papiloma virus E6 protein or adenovirus E1B gene.

In another embodiment, the invention provides a method of generating a totipotent, nearly totipotent or pluripotent stem cell homozygous for at least one MHC allele, comprising the steps of: (a) providing a differentiated cell; (b) deleting one of the two MHC alleles by gene targeting; (c) dedifferentiating said differentiated cell by reprogramming the nucleus of the cell; and (d) using loss of heterozygosity to generate a stem cell homozygous for at least one MHC allele.

In another embodiment, the invention provides a method of conducting a pharmaceutical business, comprising the steps of a) providing a stem cell line that is homozygous for at least one histocompatibility antigen, wherein said stem cell line is chosen from a bank of totipotent, nearly totipotent and/or pluripotent stem cells, comprising a library of human or non-human animal stem cells, each of which cells is hemizygous or homozygous for at least one MHC allele present in a human or non-human animal population, wherein said bank of stem cells comprise stem cells that are hemizygous or homozygous for different set of MHC alleles relative to the other members in the bank of stem cells, and wherein gene targeting or loss of heterozygosity is used to generate the hemizygous or homozygous MHC allele; and b) modifying the stem cell line to match the HLA profile of a transplant recipient. Such methods may further comprise the step of differentiating the stem cells prior to transplanting to the recipient. Methods of conducting a pharmaceutical business may also comprise establishing a distribution system for distributing the preparation for sale or may include establishing a sales group for marketing the pharmaceutical preparation.

### BRIEF DESCRIPTION OF THE DRAWINGS

FIG. 1 shows a schematic diagram of the cellular pathways that lead to the loss of heterozygosity.

FIG. 2 shows a schematic diagram displaying the modification of chromosomal gene target by homologous recombination. Homologous recombination between the gene targeting vector and its homologous chromosomal gene target produces cells with the desired gene modifications. HSV TK is the Herpes simplex virus thymidine kinase gene expression cassette conferring sensitivity to the drug Ganciclovir; Neo is the neomycin phosophotransferase gene expression cassette conferring resistance to the drug G418; DT-A is the diphtheria toxin A chain gene expression cassette; mycin is an inactive 3′ half of the puromycin acetyltransferase gene with a splice acceptor site and intron; FRT is the FLP recognition target site (FRT), LoxP is the Cre recombinase recognition sequence.

FIG. 3 shows a diagram mapping the HLA-A locus on chromosome 6p21.3 and the structure of targeting vector. A: diagrammatic map of the HLA-A locus on chromosome 6 from nucleotide 30014810 to nucleotide 30024810. For convenience, nucleotide coordinates for exon locations and gene expression cassettes will use nucleotide numbering from the indicated 10 kilobasepair scale. B: Map of the HLA-A targeting vector without the vector backbone. The expression cassette designations are the same as described in FIG. 2. DT-A is a negative selectable mammalian expression cassette for the diphtheria toxin A chain. Expression of DT-A is lethal for cells. Only cells that have undergone homologous recombination or inadvertent DT-A inactivation will survive. LoxP is the Cre recombinase recognition sequence and allow Cre mediated recombination between the tandem LoxP repeats and deletion of intervening sequences.

FIG. 4 shows a diagram displaying the deletion of a chromosomal gene target by homologous recombination with a gapped replacement targeting vector. Homologous recombination between the gapped gene targeting vector and its homologous chromosomal gene target produces cells with the desired deletion. HSV TK is the Herpes simplex virus thymidine kinase gene expression cassette conferring sensitivity to the drug Ganciclovir; Neo is the neomycin phosophotransferase gene expression cassette conferring resistance to the drug G418; mycin is an inactive 3 half of the puromycin acetyltransferase gene with a splice acceptor site and intron; FRT is the FLP recognition target site (FRT), LoxP is the Cre recombinase recognition sequence.

FIG. 5 shows a diagram mapping the HLA-C/HLA-B locus on chromosome 6p21.3 and the structure of a deletion targeting vector. At diagrammatic map of the HLA-C/HLA-B locus on chromosome 6 from nucleotide 31338716 to nucleotide 332438716. B: Map of the HLA-C/HLA-B deletion targeting vector without the vector backbone. In this vector, 90 kbp of chromosomal DNA sequences from 31343716 to 31433716 are missing, including the structural genes for HLA-C and HLA-B. The targeting vector arms each have 5 kbp homology to the chromosomal target and the targeting mechanism is illustrated in FIG. 4. A successful targeted recombinant cell line will thus be deleted for HLA-C and HLA-B. The LoxP recognition sequences are present to allow site specific recombination to remove the Neo and HSV TK expression cassettes.

FIG. 6 shows a diagram of the deletion of HLA genes by site specific recombination or I-SceI engineered deletions. The HLA-A and HLA-F genes, separated by approximately 2.2×10⁵ basepairs were modified by gene targeting to insert the LoxP, and other indicated gene sequences. Expression of the Cre recombinase catalyzes recombination between the direct LoxP repeats, deleting all of the intervening sequences and producing a cell that is missing the HSV TK gene, HLA-F, HLA-G, and HLA-A. The FRT and truncated puromycin gene remain far further site specific gene insertions.

FIG. 7 shows a diagram mapping the HLA F/HLA-A locus on chromosome 6p21.3 and structures of targeting vectors. A: diagrammatic map of the HLA-F/HLA-A locus on chromosome 6. B: Map of the HLA-F targeting vector without the vector backbone. C: Map of the HLA-A targeting vector without the vector backbone. The LoxP recognition sequences are present to allow site specific recombination to remove the Neomycin, Hygromycin, HSV TK, and GFP expression cassettes. Other cassette designation and function are described in the preceding figures.

FIG. 8 shows a diagram displaying positive selection for FLP/FRT site specific introduction of transgenes into deleted HLA genes using plug and socket site specific recombination. Gene definitions are the same as indicated in FIG. 2. Puro is the an inactive 5′ of the puromycin acetyltransferase gene. An active puromycin acetyltransferase gene is reconstructed on successful FLP mediated recombination conferring cellular resistance to puromycin.

FIG. 9 shows a diagram displaying the modification of isolated chromosomes, chromatin, or nuclei in vitro. Purified recombinase or cell free extract is shown as spheres.

FIG. 10 is a chart showing HLA types of H1, H7, H9 and H14 ES cell lines.

FIG. 11 is a chart showing the DNA sequence location of class I and class II HLA genes on human chromosome 6. Chromosome location is indicated by nucleotides and was obtained from the National Center for Biotechnology Information (NCBI) (Jun. 10, 2005 update).

FIGS. 12 A-C are charts showing the DNA sequence location of class I BLA genes. Class I HLA genes are boxed and shaded.

FIGS. 13 A-B are charts showing the DNA sequence location of class II HLA genes. Class II HLA genes are boxed and shaded.

FIG. 14 is a chart showing the chromosomal sequence location of the ABO genes (boxed and shaded).

### DETAILED DESCRIPTION OF THE INVENTION

Table of Abbreviations

| | |
|---|---|
| CT | Chromatin Transfer |
| CyT | Cytoplasmic Transfer |
| DMAP | Dimethylaminopurine |
| EC Cells | Embryonal Carcinoma Cells |
| ED Cells | Embryo-derived cells are cells |
| | derived from a zygote, blastomeres, morula or |
| | blastocyst-staged mammalian embryo produced by the |
| | fusion of a sperm and egg cell, nuclear transfer, |
| | parthenogenesis, or the reprogramming of chromatin and |
| | subsequent incorporation of the reprogrammed chromatin |
| | into a plasma membrane of an oocyte or blastomere to |
| | produce a cell line. The resulting cell line may be |
| | either a differentiated cell line or the cells may be |
| | maintained as undifferentiated ES cells. Therefore ED |
| | cells are inclusive of ES cells and cells derived by |
| | directly differentiating cells from a mammalian |
| | preimplantation embryo. |
| ES cell | Embryonic stem cells derived, |
| | e.g., from a zygote, blastomeres, morula or blastocyst- |
| | staged mammalian embryo produced by, e.g., the fusion |
| | of a sperm and egg cell, nuclear transfer, |
| | parthenogenesis, or the reprogramming of chromatin and |
| | subsequent incorporation of the reprogrammed chromatin |
| | into a plasma membrane to produce a cell. |
| hED Cells | Human embryo-derived cells are |
| | ED cells derived from a human preimplantation embryo. |
| hES Cells | human embryonic stem cells are |
| | ES cells derived from a human preimplantation embryo. |
| HSE | Human skin equivalents are |
| | mixtures of cells and biological or synthetic matrices |
| | manufactured for testing purposes or for therapeutic |
| | application in promoting wound repair. |
| ICM | Inner cell mass of the |
| | mammalian blastocyst-stage embryo. |
| MiRNA | Micro RNA |
| NT | Nuclear Transfer |
| ps fibroblasts | Pre-scarring fibroblasts |
| | are fibroblasts derived from the skin of early |
| | gestational skin or derived from ED cells that display |
| | a prenatal pattern of gene expression with that they |
| | promote the rapid healing of dermal wounds without scar |
| | formation. |
| RCL | Reduced Complexity Library |
| SCNT | Somatic Cell Nuclear Transfer |
| SPF | Specific Pathogen-Free |
| LOH | loss of heterozygosity |

### DEFINITIONS

The term “cellular reconstitution” refers to the transfer of a nucleus or chromatin to cellular cytoplasm so as to obtain a functional cell.

The term “chromatin transfer” (CT) refers to the cellular reconstitution of condensed chromatin.

The term “condensed chromatin” refers to DNA not enclosed by a nuclear envelope. Condensed chromatin my result, for example, by exposing a nucleus to a mitotic extract such as from an M1 or an MII oocyte or other mitotic cell extract, by transferring a nucleus into an M1 or an MII oocyte or other mitotic cell and retrieving the resulting condensed chromatin following the breakdown of the nuclear envelop. Condensed chromatin refers to chromosomes that are in a greater degree of compaction than occurs in any phase of the cell cycle other than metaphase.

The term “cytoplasmic bleb” refers to the cytoplasm of a cell bound by an intact, or permeabilized, but otherwise intact plasma membrane but lacking a nucleus. It is used interchangeably and synonymously with the term “anucleate cytoplast” and “anuceate cytoplasm” unless the term “anucleate cytoplasm” is explicitly used in the context of an extract in which the plasma membrane has been removed.

The term “cytoplasmic transfer” (CyT) refers to any number of techniques known in the art for juxtaposing the nucleus for genome) of a somatic cell with the cytoplasm of an undifferentiated cell. Such techniques include, but are not limited to, the direct transfer (by, for example, microinjection) of said undifferentiated cytoplasm into the cytoplasm of a differentiated cell, the permeabilization of a somatic cell and exposure to undifferentiated cell cytoplasm or extracts of undifferentiated cells, or the transfer of the somatic cell nucleus into a cytoplasmic bleb of an undifferentiated cell.

The term “differentiated cell” refers to any cell from any vertebrate species in the process of differentiating into a somatic cell lineage or having terminally differentiated into the type of cell it will be in the adult organism.

The term “pluripotent stem cells” refers to animal cells capable of differentiating into more than one differentiated cell type. Such cells include ES cells, EG cells, EDCs, ED-like cells, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification within the egg.

The term “embryonic stem cells” (ES cells) refers to cells derived from the inner cell mass of blastocysts or morulae that have been serially passaged as cell lines or embryonic stem cells derived from other sources. The ES cells may be derived from fertilization of an egg cell with sperm or DNA, nuclear transfer, parthenogenesis, or by means to generate hES cells with homozygosity in the MHC region.

The term “human embryonic stem cells” (hES cells) refers to cells derived from the inner cell mass of human blastocysts or morulae that have been serially passaged as cell lines or human embryonic stem cells derived from other sources. The hES cells may be derived from fertilization of an egg cell with sperm or DNA, nuclear transfer, parthenogenesis, or by means to generate hES cells with homozygosity in the HLA region.

The term “fusigenic compound” refers to a compound that increases the likelihood that a condensed chromatin or nucleus is fused with and incorporated into a recipient cytoplasmic bleb resulting in a viable cell capable of subsequent cell division. Such fusigenic compounds may, by way of nonlimiting example, increase the affinity of a condensed chromatin or a nucleus with the plasma membrane. Alternatively, the fusigenic compound may increase the likelihood of the joining of the lipid bilayer of the target cytoplasmic bleb with the condensed chromatin, nuclear envelope of an isolated nucleus, or the plasma membrane of a donor cell.

The term “heteroplasmon” refers to a cell resulting from the fusion of a cell containing a nucleus and cytoplasm with the cytoplast of another cell.

The term “human embryo-derived cells” (hEDC) refer to morula-derived cells, blastocyst-derived cells including those of the inner cell mass, embryonic shield, or epiblast, or other totipotent or pluripotent: stem cells of the early embryo, including primitive endoderm, ectoderm, and mesoderm and their derivatives, but excluding hES cells that have been passaged as cell lines. The hEDC cells may be derived from fertilization of an egg cell with sperm or DNA, nuclear transfer, parthenogenesis, or by means to generate hES cells with homozygosity in the HLA region.

The term “human embryo-derived-like cells” (hED-like) refer to pluripotent stem cells produced by the present invention that are not cultured so as to retain the characteristics of ES cells, but like morula-derived cells, blastocyst-derived cells including those of the inner cell mass, embryonic shield, or epiblast, or other totipotent or pluripotent stem cells of the early embryo, including primitive endoderm, ectoderm, and mesoderm and their derivatives that have not been cultured so as to maintain stable hES lines, are capable of differentiating into any of the somatic cell differentiated types. The hED-like cells may be derived with genetic modifications, including modified so as to lack genes of the MHC region, to be hemizygous or homozygous in this region.

The term “nuclear remodeling” refers to the artificial alteration of the molecular composition of the nuclear lamina or the chromatin of a cell.

The term “permeabilization” refers to the modification of the plasma membrane of a cell such that there is a formation of pores enlarged or generated in it or a partial or complete removal of the plasma membrane.

The term “pluripotent” refers to the characteristic of a stem cell that said stem cell is capable of differentiating into a multitude of differentiated cell types.

The term “reduced complexity library” or “RCL” refers to a collection of cells or animals with MHC genes altered in a form that results in cells or animals with cells or tissues with a greater potential to be transplanted into another animal without rejection that the average random sample of wild-type cells or tissues would undergo.

The term “inducible suicide gene” refers to any genetic modification of a cell that results in a cell that can be induced to undergo cell death or can be induced to express a cell surface protein that would lead to the death or removal of said cell from en organism or from a cell culture system. For example, a suicide gene that is induced in a cell may cause a host animal to recognize the cell and attack it with a host immune response, such immune response being, for example, cell-mediated or mediated by antibody and complement. Alternatively, a suicide gene may result in the death of the cell in response to external stimuli.

The term “totipotent” refers to the characteristic of a stem cell that said stem cell is capable of differentiating into any cell type in the body.

The term “undifferentiated cell” refers to the cytoplasm of an oocyte, an undifferentiated cell such as an ES, EG, ICM, ED, EC, teratocarcinoma cell, blastomere, morula, or germ-line cell.

1. Overview

The present invention provides totipotent, nearly totipotent, and/or pluripotent stem cell lines that are hemizygous or homozygous for one or more Histocompatibility antigen genes, such as, for example, in the case of human stem cells and “stem-like” cells, MHC genes that are present in the human population. In certain embodiments, these stem cell lines are hemizygous or homozygous for MHC alleles that are representative of at least the most prevalent in the particular species, the most preferred species being human. In the context of this invention, cell lines that are homozygous for one or more Histocompatibility antigen genes include cell lines that are nullizygous for one or more (preferably all) such genes. Nullizygous for a genetic locus means that the gene is null at that locus, i.e., both alleles of that gene are deleted or inactivated. Stem cells that are nullizygous for all MHC genes may be produced by standard methods known in the art, such as, for example, gene targeting and/or LOH.

In certain embodiments, the lines of the present invention also have an ABO blood group type O-negative to make them broadly compatible across the different blood types. The ABO blood antigens play a role in rejection of not only blood cells in transfusions, but of some tissue cells as well. In addition, O-derived blood cells are universal in application. The stem cell lines described herein may be derived from a male or a female. Preferably, the stem cell lines are derived from a female.

The stem cells made by and used for the methods of the present invention may be any appropriate totipotent, nearly totipotent, or pluripotent stem cells. Such cells include, for example, inner cell mass (ICM) cells, embryonic stem (ES) cells, embryonic germ (EG) cells, embryos consisting of one or more cells, embryoid body (embryoid) cells, morula-derived cells, as well as multipotent partially differentiated embryonic stem cells taken from later in the embryonic development process, and also adult stem cells, including but not limited to nestin positive neural stem cells, mesenchymal stem cells, hematopoietic stem cells, pancreatic stem cells, marrow stromal stem cells, endothelial progenitor cells (PCs), bone marrow stem cells, epidermal stem cells, hepatic stem cells and other lineage committed adult progenitor cells.

Totipotent, nearly totipotent, or pluripotent stem cells, and cells therefrom, for use in the present invention can be obtained from any sources of such cells. One means for producing totipotent, nearly totipotent, or pluripotent stem cells, and cells therefrom, for use in the present invention is via nuclear transfer into a suitable recipient cell as described, for example, in U.S. Pat. No. 5,945,577, and U.S. Pat. No. 6,215,041, the disclosures of which are incorporated herein by reference in their entirety. Nuclear transfer using an adult differentiated cell as a nucleus donor facilitates the recovery of transfected and genetically modified stem cells as starting materials for the present invention, since adult cells are often more readily transfected than embryonic cells.

Stem cell lines of the present invention can be induced to differentiate into cell types suitable for therapeutic transplant. Because the cells of the present invention have hemizygous or homozygous MHC alleles, the chance of obtaining cells for transplant that have MHC alleles that match those of a patient in need of a transplant is significantly enhanced. Instead of having to find a six of six match between two sets of HLA-A, HLA-B, and HLA-DR antigens, a high level of Histocompatibility is provided by the cells for transplant of the present invention when either of the two HLA-A, HLA-B, and HLA-DR antigens of the prospective transplant recipient matches one of the corresponding hemizygous or homozygous HLA antigens of the cells for transplant. In one embodiment, the invention provides a bank of stem cells comprising hemizygous or homozygous MHC alleles. Stem cell lines that are hemizygous or homozygous at the MHC locus are advantageous because fewer stem cell lines are needed to match the HLA genes to those of a transplant recipient. For example, only 72 stem cell lines that are hemizygous or homozygous at the MHC locus are required to match a patient; whereas a bank of stem cells with heterozygous HLA-A and HLA-B antigens would need to have 4032 different stern cell lines. To provide a library of heterozygous stem cell lines that match the WHO list of serological types would require obtaining stem cells having every combination of 28 different pairs of HLA-A antigens and 59 different Pairs of HLA-B, to account for both the maternal and paternal alleles for each loci. The complexity of such a stem cell bank, i.e., the number of different cell lines required, would be 2,567,032. In contrast, a bank of stem cells hemizygous or homozygous for the same HLA-A and HLA-B antigens would only need to have a complexity of 1,652 stem cell lines to guarantee a match to a patient with HLA-A and -B antigens on the WHO list of serological types. The actual number required to meet the needs of a majority of patients will actually be less than this due to the non-random distribution of alleles in various populations around the world. Patients in need of bone marrow stem cell grafts who are homozygous in particular alleles are particularly sensitive to graft versus host disease when heterozygous bone marrow grafts are used. Stem cell grafts using stem cells having homozygous alleles made according to the methods of the present invention would alleviate this common complication of transplants.

The present invention provides novel means for making totipotent and/or pluripotent stem cells that can serve as sources of cells for therapeutic transplant that are highly histocompatible with human or non-human patients in need of cell transplants. Such cell lines are useful in creating animal models for specific diseases that may be used to evaluate potential treatments and drug antidotes, or may be useful for other veterinary purposes. A variety of non-human animals may be treated according to the present invention, including primates, horses, dogs, cats, pigs, goats, and cows.

In one embodiment, the invention comprises preparing stem cell lines that are hemizygous or homozygous for one or more critical Histocompatibility antigen alleles, in the case of human stem cells. Homozygous or hemizygous stem cell lines may be matched for any transplant recipient, and may comprise MHC alleles that are present in all or most of the world's populations, including the populations of North America, Central and South America, Europe, Africa, Oceania, Asia, and the Pacific islands. It is an object of the present invention to provide stem cells generated from any cell that is hemizygous or homozygous for one or more critical antigen alleles. A variety of mammalian cells may be used in the invention, including but not limited to, ES, EG, ED, pluripotent stem cells, or differentiated somatic cells from human or non-human animals.

The stem cell lines of the present invention comprise lines of totipotent, nearly totipotent, and/or pluripotent stem cells that are hemizygous or homozygous for at least one Histocompatibility antigen collection. In the case of human stem cells this will be an MHC allele selected from the group consisting of HLA-A, HLA-B, HLA-C, HLA-DR, HLA-DQ, and HLA-DP. In one embodiment, the stem cell bank comprises totipotent, nearly totipotent, and/or pluripotent stem cells that are hemizygous or homozygous for the significant Histocompatibility antigen alleles, e.g., the HLA-A, HLA-B, and HLA-DR alleles. In another embodiment, the stem cell lines comprise stem cells that are hemizygous or homozygous for all of the Histocompatibility antigen alleles, e.g., MHC alleles.

The stem cell bank of the present invention comprises totipotent and/or nearly totipotent stem cells such as embryonic stem (ES) cells, that can differentiate in vivo or ex viva into a wide variety of different cell types having one or more hemizygous or homozygous MHC alleles. The stem cell lines of the present invention can also comprise partially differentiated, pluripotent stem cells such as neuronal stem cells and/or hematopoietic stern cells, that differentiate in vivo or ex vivo into a more limited number of differentiated cell types having one or more homozygous MHC alleles. These stem cells may comprise a heterologous gene (i.e., be transgenic) they may express antigens that encode therapeutic or diagnostic proteins and polypeptides. For example, the stem cells may be genetically engineered to express proteins that inhibit immune rejection responses such as CD40-L (CD154 or gp39) or in the case of porcine stem cells may be genetically engineered to knock-out a glycosylated antigen that is known to trigger immune rejection responses.

In one embodiment, the present invention provides a stem cell bank comprising stem cells having hemizygous or homozygous Histocompatibility alleles, such as MHO alleles, that are available “off the shelf” for providing histocompatible cells suitable for transplant to patients in need thereof. Desirably, this stem cell bank will include stem cell lines that are representative of the different Histocompatibility antigens expressed in the particular species, such as human. In one embodiment, the stem cell bank comprises stem cells that are isolated and maintained without feeder cells or serum of non-human animals, to minimize concerns of contamination by pathogens. In another embodiment, the stem cell bank comprises stem cells that are genetically modified relative to the cells of the donor. In certain embodiments, the stem cell bank may comprise stem cells that are genetically modified with an inducible suicide gene or genes to remove the cells from a culture by inducing cell death, or to remove the cells from an animal or human when the cells are no longer desired or where their presence endangers the health of said animal or human. This invention also provides such stem cells as part of a bank or not. In another embodiment, HLA genes are modified to make an HLA null stem cell line, or numerous different hemizygous or homozygous HLA cell lines with an otherwise common or essentially common genotype that reduces the variations in culture conditions commonly observed between different cell lines, such as different human ES cell lines. In another embodiment of the invention, a cell line with an inducible suicide gene or genes is modified to make an HLA null stem cell line, or numerous different hemizygous or homozygous HLA cell lines with an otherwise common or essentially common genotype. In another embodiment, the stem cell bank comprises stem cells generated through the reprogramming of differentiated cells (e.g., somatic cells) by exposure to the cytoplasm of undifferentiated cells. In another embodiment, the stem cell bank comprises stem cells generated by nuclear transfer techniques that are rejuvenated, or “hyper-youthful,” relative to the cells of the donor, and also relative to age-matched control cells of the same type and species that are not generated by nuclear transfer techniques. Such rejuvenated or “hyper-youthful” cells have extended telomeres, increased proliferative life-span, and metabolism that is more characteristic of youthful cells, having, for example, increased EPC-1 and telomerase activities, relative to the donor cells from which they are derived, and also relative to age-matched control cells of the same type that are not generated by nuclear transfer techniques. In certain embodiments, the donor is a non-human mammal or a human. In a preferred embodiment, the donor is human.

This invention also provides stem cells that have been genetically modified with an inducible suicide gene or genes to remove the cells from a culture by inducing cell death, or to remove the cells from an animal or human when the cells are no longer desired or where their presence endanger the health of said animal or human; preferably the stem cells are O-negative, preferably the stem cells are from a female (i.e., female stem cells such as female ES cells). In certain embodiments, the stem cells described in the preceding sentence further have their HLA genes modified to make HLA null stem cell line (s), or numerous different hemizygous or homozygous HLA cell lines with an otherwise common or essentially common genotype that reduces the variations in culture conditions commonly observed between different cell lines, such as different human ES cell lines. In certain embodiments, the stem cells with the same inducible suicide gene or genes are made into HLA null stem cells by, for example, gene targeting or by LOH, and then different HLA alleles are added back to different cells of this population of cells to make a set of hemizygous HLA lines, each of which otherwise has the same genotype and same suicide gene(s) sequence. In another embodiment of the invention, a stem cell line with an inducible suicide gene or genes is modified to make an HLA null stem cell line, or numerous different hemizygous or homozygous HLA cell lines with an otherwise common or essentially common genotype.

Another object of the invention is to provide a method by which a human or non-human animal in need of a cell or tissue transplant could be provided with cells or tissue suitable for transplantation that have hemizygous or homozygous Histocompatibility antigen alleles. In certain embodiments, in the case of human recipients, MHC alleles that match the MHC alleles of the transplant recipient. The invention provides a method in which the MHC alleles of a transplant recipient are identified, and a line of stem cells homozygous for at least one MHC allele present in the recipient's cells is obtained from a stem cell bank produced according to the disclosed methods. That line of stem cells is then used to generate cells or tissue suitable for transplant that are homozygous for at least one MHC allele present in the recipient's cells. The method of the present invention further comprises grafting the cells or tissue of this invention to the body of the transplant recipient. In one embodiment of the invention, three, four, five, six or more of the MHC alleles of the line of stem cells used to generate cells or tissue for transplant are homozygous and match MHC alleles of the transplant recipient.

In one embodiment, the line of stem cells used to generate cells or tissue suitable for transplant is a line of totipotent or nearly totipotent embryonic stem cells. In another embodiment, the line of stem cells used to generate cells or tissue suitable for transplant is a line of hematopoietic stem cells. The lines of stem cells that can be used to generate cells or tissue suitable for transplant may be available “off the shelf” in the stem cell bank of the present invention. In one embodiment, the stem cell bank of the present invention comprises lines of totipotent, nearly totipotent, and/or pluripotent stem cells that are lines of rejuvenated, “hyper-youthful cells” generated by nuclear transfer techniques. In another embodiment, the stem cell, bank of the present invention comprises one or more lines of totipotent, nearly totipotent, and/or pluripotent stem cell having DNA that is genetically modified relative to the DNA of the human donor from which they are derived. For example, the invention comprises altering genomic DNA of the cells to replace a non-homozygous MHC allele with one that is hemizygous or homozygous, or to inhibit the effective presentation of a class I or class II HLA antigen on the cell surface, e.g., by preventing expression of beta2-microglobulin, or by preventing expression of one or more MHC alleles. Also, the invention encompasses introducing one or more genetic modifications that result in lineage-defective stem cells, i.e., stem cells which cannot differentiate into specific cell lineages.

In another embodiment, the invention provides a method of conducting a pharmaceutical business, comprising: a) providing a stem cell line that is homozygous for at least one Histocompatibility antigen (said line could be part of a bank of cell lines); and, b) modifying the stem cell line to match the HLA profile of a transplant recipient. The method may further comprise differentiating the stem cells prior to transplant to the recipient. Preferably, the method of conducting a pharmaceutical business includes an additional step of establishing a distribution system for distributing the preparation for sale, and (optionally) establishing a sales group for marketing the pharmaceutical preparation.

In another embodiment, this invention provides a method of conducting a pharmaceutical business, comprising the steps of providing regional centers that bank cryopreserved pluripotent stem cells with reduced complexity to a clinical center where they are differentiated into a therapeutically-useful cell type, or where the differentiation is performed earlier and the cells are banked in the regional center and the cells ready for transplantation are shipped in live cultures or in a cryopreserved state to the health care provider.

In another embodiment, this invention provides methods that comprise the utilization of cells with reduced complexity (RCL) in the MHC genes in research and in therapy. Such RCL cells may be pluripotent or totipotent cells and may be differentiated into any of the cells in the body including, without limitation, skin, cartilage, bone skeletal muscle, cardiac muscle, renal, hepatic, blood and blood forming, vascular precursor and vascular endothelial, pancreatic beta, neurons, glia, retinal, inner ear follicle, intestinal, or respiratory cells.

In certain embodiments, the reprogrammed cells may be differentiated into cells with a dermatological prenatal pattern of gene expression that is highly elastogenic or capable of regeneration without causing scar formation. Dermal fibroblasts of mammalian fetal skin, especially corresponding to areas where the integument benefits from a high level of elasticity, such as in regions surrounding the joints, are responsible for synthesizing de novo the intricate architecture of elastic fibrils that function for many years without turnover. In addition, early embryonic skin is capable of regenerating without scar formation. Cells from this point in embryonic development made from the reprogrammed cells of the present invention are useful in promoting scarless regeneration of the skin including forming normal elatin architecture. This is particularly useful in treating the symptoms of the course of normal human aging, or in actinic skin damage, where there can be a profound elastolysis of the skin resulting in an aged appearance including sagging and wrinkling of the skin.

In another embodiment of the invention, the reprogrammed cells are exposed to inducers of differentiation to yield therapeutically useful cells such as retinal pigment epithelium, hematopoietic precursors and hemangioblastic progenitors as well as many other useful cell types of the endoderm, mesoderm, and endoderm. Such inducers include, but are not limited to: cytokines such as interleukin-alpha A, interferon-alpha A/D, interferon-beta, interferon-gamma, interferon-gamma-inducible protein-10, interleukin-1-17, keratinocyte growth factor, leptin, leukemia inhibitory factor, macrophage colony-stimulating factor, and macrophage inflammatory protein-1 aloha, 1-beta, 2, 3 alpha, 3 beta, and monocyte chemotactic protein 1-3, 6kine, activin A, amphiregulin, angiogenin, B-endothelial cell growth factor, beta cellulin, brain-derived neurotrophic factor, C10, cardiotrophin-1, ciliary neurotrophic factor, cytokine-induced neutrophil chemoattractant-1, eotaxin, epidermal growth factor, epithelial neutrophil activating peptide-78, erythropioetin, estrogen receptor-alpha, estrogen receptor-beta, fibroblast growth factor (acidic and basic), heparin, FLT-3/FLK-2 ligand, glial cell line-derived neurotrophic factor, Gly-His-Lys, granulocyte colony stimulating factor, granulocytemacrophage colony stimulating factor, GRO-alpha/MGSA, GRO-beta, GRO-gamma, HCC-1, heparin-binding epidermal growth factor, hepatocyte growth factor, heregulin-alpha, insulin, insulin growth factor binding protein-1, insulin-like growth factor binding protein-1, insulin-like growth factor, insulin-like growth factor II, nerve growth factor, neurotophin-3,4, oncostatin M, placenta growth factor, pleiotrophin, rantes, stem cell factor, stromal cell-derived factor 1B, thromopoietin, transforming growth factor-(alpha, beta1, 2, 3, 4, 5), tumor necrosis factor (alpha and beta), vascular endothelial growth factors, and bone morphogenic proteins, enzymes that alter the expression of hormones and hormone antagonists such as 17B-estradiol, adrenocorticotropic hormone, adrenomedullin, alpha-melanocyte stimulating hormone, chorionic gonadotropin, corticosteroid-binding globulin, corticosterone, dexamethasone, estriol, follicle stimulating hormone, gastrin 1, glucagons, gonadotropin, L-3,3′,5′-triiodothyronine, leutinizing hormone, L-thyroxine, melatonin, MZ-4, oxytocin, parathyroid hormone, PEC-60, pituitary growth hormone, progesterone, prolactin, secretin, sex hormone binding globulin, thyroid stimulating hormone, thyrotropin releasing factor, thyroxin-binding globulin, and vasopressin, extracellular matrix components such as fibronectin, proteolytic fragments of fibronectin, laminin, tenascin, thrombospondin, and proteoglycans such as aggrecan, heparan sulphate proteoglycan, chontroitin sulphate proteoglycan, and syndecan. Other inducers include cells or components derived from cells from defined tissues used to provide inductive signals to the differentiating cells derived from the reprogrammed cells of the present invention. Such inducer cells may be derived from a human, a nonhuman mammal, or an avian, such as specific pathogen-free (SPF) embryonic or adult cells.

2. Hemizygous and Homozygous Cell Lines by Gene Targeting and/or Loss of Heterozygosity

The invention provides two complementary approaches that when used together may generate cells that are hemizygous or homozygous for one, a portion of, or all of the genes in the MHC complex of a cell. A variety of mammalian cells may be used in the invention, including but not limited to, ES, EG, ED, pluripotent stem cells, or differentiated somatic cells from human or non-human animals. In one embodiment, the invention provides a mammalian cell that comprises modifications to one of the alleles of sister chromosomes in the cell's MHC complex. A variety of methods for generating gene modifications, such as gene targeting, may be used to modify the genes in the MHC complex. In a further embodiment, the modified alleles of the MHC complex in the cells described herein are subsequently engineered to be homozygous so that identical alleles are present on sister chromosomes. Methods such as LOH may be utilized in the invention to engineer cells to have homozygous alleles in the MHC complex. For example, one or more genes in a set of MHC genes from a parental allele can be targeted to generate hemizygous cells. The other set of MHC genes can be removed by gene targeting or LOH to make a null line. This null line can be used further, for example in stem cell therapy, or it can be used as the host cell line in which to drop arrays of the HLA genes, or individual genes, to make a hemizygous bank with an otherwise uniform genetic background.

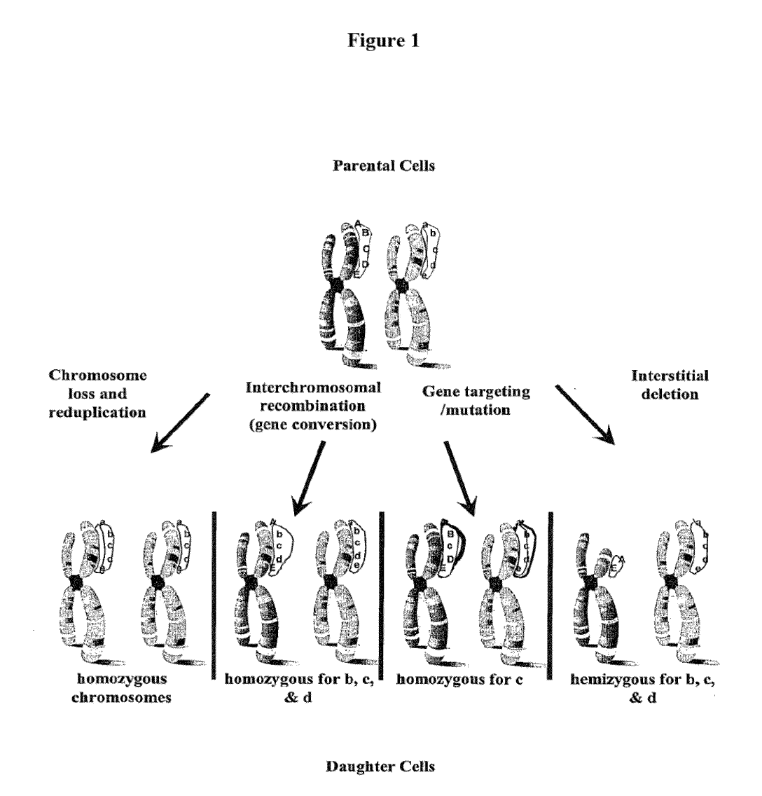

Gene targeting has successfully been used to engineer defined chromosomal gene modifications in mouse ES cell lines, hES cell lines and other rodent and human cell lines. While this approach is well established, it is labor intensive and cannot readily be used for the simultaneous modification of the two alleles of sister chromosomes. LOH is a complementary approach that can be used to generate cells homozygous for a gene allele or homozygous for a gene targeted allele. LOH, or Loss of Heterozygosity, is the loss of one functional allele or haplotype thus leaving the cell with one remaining haplotype. LOH can generate a “uni” haplotype for individual genes, gene clusters, or entire chromosomes depending on the underlying molecular mechanism for the LOH (FIG. 1).

A. LOH for Engineering MHC Genes in Human Embryonic Stem Cells

Several molecular mechanisms are now known to cause LOH in mitotically dividing cells (FIG. 1). LOH is often observed in cancer cells where one copy of a gene, or closely linked genes, is missing and which is believed in many cases to be an early initiating event causing or contributing to uncontrolled cell growth. LOH from loss of en entire chromosome, believed to result from chromosomal nondisjunction, followed by reduplication of the remaining chromosome can produce diploid cells with uniparental disomy homozygous for that entire chromosomal genetic complement (Campbell and Worton, Mol Cell Biol 1:336-346 (1981), Eves and Farber, Proc Natl Acad Sci USA 78:1768-1772 (1981), Turner, et al., Proc Natl Acad Sci USA 85:3189-3192 (1988), de Nooij-van Dalen, et al., Mutat Res 374:51-62 (1997), de Nooij-van Dalen, et al., Genes Chromosomes Cancer 21:30-38 (1998), de Nooij-van Dalen, et al., Genes Chromosomes Cancer 30:323-335 (2001), Cervantes, et al., Proc Natl Acad Sci USA 99:3586-3590 (2002)). LOH can also be due to interstitial deletions resulting in chromosomes hemizygous for the deleted loci leaving behind one parental gene copy (Eves and Farber, Proc Natl Acad Sci USA 78:1768-1772 (1981), Turner, et al., Proc Natl Acad Sci USA 85:3189-3192 (1988), Adair, et al., Mutat Res 72:187-205 (1980), Bradley and Letovanec, Somatic Cell Genet 8:51-66 (1932), Simon, et al., Mol Cell Biol 2:1126-1133 (1982), Adair and Carver, Environ Mutagen 5:161-175 (1983), Adair, et al., Proc Natl Acad Sci USA 80:5961-5964 (1983), Bradley, Mol Cell Biol 3:1172-1181 (1983), Simon and Taylor, Proc Natl Acad Sci USA 80:810-814 (1983), Koufos, et al., Nature 316:330-334 (1985), Harwood, et al., Hum Mol Genet 2:165-171 (1993)). While interstitial deletions do not generate true diploid homozygosity, the cells in this case are functionally homozygous since one gene allele is missing. LOH may also be due to interchromosomal homologous recombination events where gene conversion results in homozygosity over several genetic loci (Campbell and Worton, Mol Cell Biol 1:336-346 (1981), Turner, et al., Proc Natl Acad Sci USA 85:3189-3192 (1988), de Nooij-van Dalen, et al., Mutat Res 374:51-62 (1997), de Nooij-van Dalen, et al., Genes Chromosomes Cancer 21:30-38 (1998), de Noon-van Dalen, et al., Genes Chromosomes Cancer 30:323-335 (2001), Gupta, et al., Cancer Res 57:1188-1193 (1997), Gupta, et al., Cytogenet Cell Genet 76:214-218 (1997), de Nooij-van Dalen, et al., Mutat Res 423:1-10 (1999), Shao, et al., Proc Natl Acad Sci USA 96:9230-9235 (1999), Shao, et al., Proc Natl Acad Sci USA 97:7405-7410 (2000), Shao, et al., Nat Genet 28:169-172 (2001)). Unlike the cases where LOH generates uniparental disomy or arises from interstitial deletions generating hemizygous chromosomes, interchromosomal recombination leaves both parental chromosomes intact, albeit homozygous over only a portion of the chromosomes. LOH due to point mutations and smaller gene rearrangements appear to be relatively rare (Simon, et al., Mol Cell Biol 2:1126-1133 (1982), Adair, et al., Proc Natl Acad Sci USA 80:5961-5964 (1983), Simon and Taylor, Proc Natl Acad Sci USA 80:810-814 (1983), Simon, et al., Mol Cell Biol 3:1703-1710 (1983)).

The frequency of LOH and underlying LOH mechanisms chromosomal loss, interstitial deletion, interchromosomal recombination, or point mutation) may vary with the cell and tissue type. For example, LOH occurs naturally at frequencies varying from approximately 1×10⁻⁷ to 1×10⁻⁴ with a median frequency of approximately 1×10⁻⁵ in mitotically dividing cells in tissue culture and in the tissues of living organisms (Turner, et al., Proc Natl Acad Sci USA 85:3189-3192 (1988), de Nooij-van Dalen, et al., Mutat Res 374:51-62 (1997), de Nooij-van Dalen, et al., Genes Chromosomes Cancer 21:30-38 (1998), de Nooij-van Dalen, et al., Genes Chromosomes Cancer 30:323-335 (2001), Cervantes, et al., Proc Natl Acad Sci USA 99:3586-3590 (2002), Simon, et al., Mol Cell Biol 2:1126-1133 (1982), Adair, et al., Proc Natl Acad Sci USA 80:5961-5964 (1983), Bradley, Mol Cell Biol 3:1172-1181 (1983), Gupta, et al., Cancer Res 57:1188-1193 (1997), de Nooij-van Dalen, et al., Mutat Res 4231-10 (1999), Shao, et al., Proc Natl Acad Sci USA 95:9230-9235 (1999), Shao, et al., Nat Genet 28:169-172 (2001), Pious, et al., Proc Natl Acad Sci USA 70:1397-1400 (1973), Janatipour, et al., Mutat Res 198:221-226 (1988), Hakoda, et al., Cancer Res 50:1738-1741 (1990), Mortensen, et al., Mol Cell Biol 12:2391-2395 (1992), Lefebvre, et al., Nat Genet 27:257-258 (2001), Sharma, et al., Transplantation 75:430-436 (2003), Kolber-Simonds, et al., Proc Natl Acad Sci USA 101:7335-7340 (2004)). In the mouse, the frequency of LOH in mouse ES cells is approximately 2×10⁻⁷, whereas the frequency of LOH in Mouse Embryonic Fibroblast (MEF) cells is approximately 100-fold higher (Cervantes, et al., Proc Natl Acad Sci USA 99:3586-3590 (2002)). LOH due to chromosomal loss duplication in mouse ES cells accounts for 57% of the LOH events with 41% of the LOH events due to mitotic recombination (Cervantes, et al., Proc Natl Acad Sci USA 99:3586-3590 (2002)). In contrast, 100% of LOH products in MEP cells are apparently due to somatic recombination (Cervantes, et al., Proc Natl Acad. Sci USA 99:3586-3590 (2002)). Similarly, recombination and chromosome loss/duplication appear to account for the bulk of LOH in human lymphoblast cell lines (Turner, et al., Proc Natl Acad Sci USA 85:3189-3192 (1988), de Nooij-van Dalen, et al., Mutat Res 374:51-62 (1997), de Nooij-van Dalen, et al., Genes Chromosomes Cancer 2130-38 (1998), de Nooij-van Dalen, et al., Genes Chromosomes Cancer 30:323-335 (2001), Gupta, et al., Cancer Res 57:1188-1193 (1997), de Nooij-van Dalen, et al., Mutat Res 423:1-10 (1999), Shao, et al., Nat Genet 28:169-172 (2001), Janatipour, et al., Mutat Res 198:221-226 (1988), Hakoda, et al., Cancer Res 50:1738-1741 (1990)). In Chinese Hamster Ovary (CHO) cells and for many cancer cell lines, however, the most frequently recovered LOH products are gene rearrangements, presumably due to large deletions, generating large regions of chromosomal hemizygosity (Simon, et al., Mol Cell Biol 2:1126-1133 (1982), Adair, et al., Proc Natl Acad Sci USA 80:5961-5964 (1983), Bradley, Mol Cell Biol 3:1172-1181 (1983), Harwood, et al., Hum Mol Genet 2:165-171 (1993)). Accordingly, the prevailing LOH products due to chromosome loss/duplication and recombination in a variety of cell types supports the idea that LOH can be used to generate stem cells functionally homozygous for targeted genes and chromosomes.

LOH has been used to create cell lines homozygous for gene knockouts in mice and pigs. LOH was used to generate mouse ES cells homozygous for genes that were modified by gene knockouts with the neomycin resistance gene by selection in high levels of G418 (Mortensen, et al., Mol Cell Biol 12:2391-2395 (1992), Lefebvre, et al., Nat Genet 27257-258 (2001)). This approach was possible because cell survival in high G418 concentrations in culture is dependent on the intracellular levels of the protein encoded by the neomycin resistance gene, Homozygous GalT knockouts in pig primary fibroblasts were generated by negatively selecting primary pig fibroblasts using GalT antisera with complement mediated cell killing to produce cells for nuclear transfer to generate GalT null pigs. In this strategy, pig fibroblasts homozygous for the GalT knockouts were enriched through serial negative selections (Sharma, et al., Transplantation 75:430-436 (2003), Kolber-Simonds, et al., Proc Natl Acad Sci USA 101:7335-7340 (2004)). While the mechanism for LOH for the positively selected G418 mouse ES cells appears to be chromosome loss/duplication (Lefebvre, et al., Nat Genet 27:257-258 (2001)), several LOH chromosomal products were identified for the negative GalT selections including interstitial deletion and homologous recombination (Kolber-Simonds, et al., Proc Natl Acad Sci USA 101:7335-7340 (2004)). This may be due to the difference in selection, strategies employed. For positive selections, there are a limited number of LOH outcomes that could lead to cells homozygous for the gene knockout.

In one aspect, the invention provides a bank of ES cell lines, wherein each member of the bank is homozygous for at least one HLA gene. This avoids the long and labor intensive process of producing hES cell lines from each individual patient and the differentiation of these cells into the required tissue for therapy. Because LOH often is due to more than one mechanism, it should be possible to recover cells that are homozygous or hemizygous for specific HLA antigens. In another aspect, the invention provides HLA-matched cells and tissues, wherein a line of ES cells is selected and expanded from a cell bank. This line of HLA-matched cells and tissues may be used for a patient in need of a cell transplant.

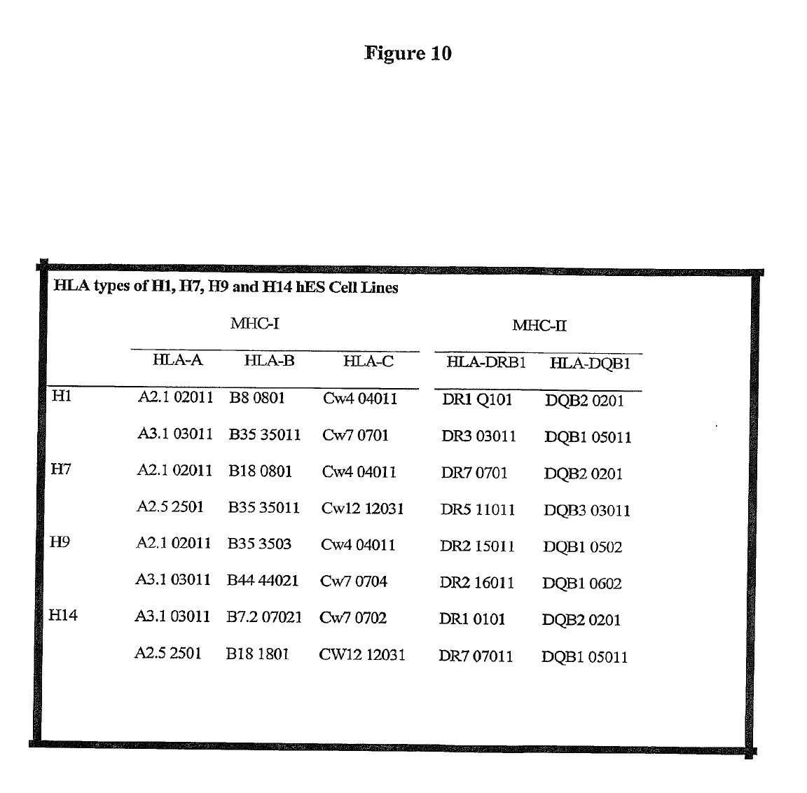

HLA specific antisera with complement mediated cell killing has previously been used to isolate cells expressing only one HLA haplotype. (Turner, et al., Proc Natl Acad Sci USA 85:3189-3192 (1988), de Nooij-van Dalen, et al., Mutat Res 374:51-62 (1997), de Nooij-van Dalen, et al., Genes Chromosomes Cancer 21:30-38 (1998), de Nooij-van Dalen, at Genes Chromosomes Cancer 30:323-335 (2001), de Nooij-van Dalen, et al., Mutat Res 423:1-10 (1999), Pious, at al., Proc Natl Acad Sci USA 70:1397-1400 (1973), Janatipour, et al., Mutat Res 198:221-226 (1988)). As described above, recombination and chromosome loss/duplication appear to account for the bulk of LOH at the HLA loci in human lymphoblast cell lines and in mouse ES cells. Some of the HLA types for the hES cell lines H1, H7, H9, and H14 are identified in FIG. 10.

B. Gene Targeting to Enable LOH on Chromosomes

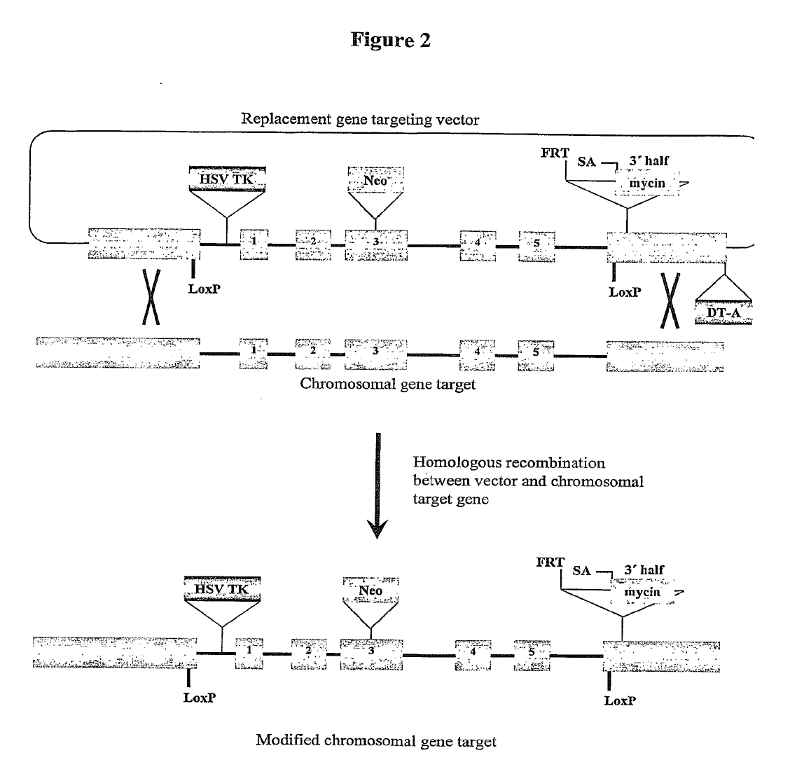

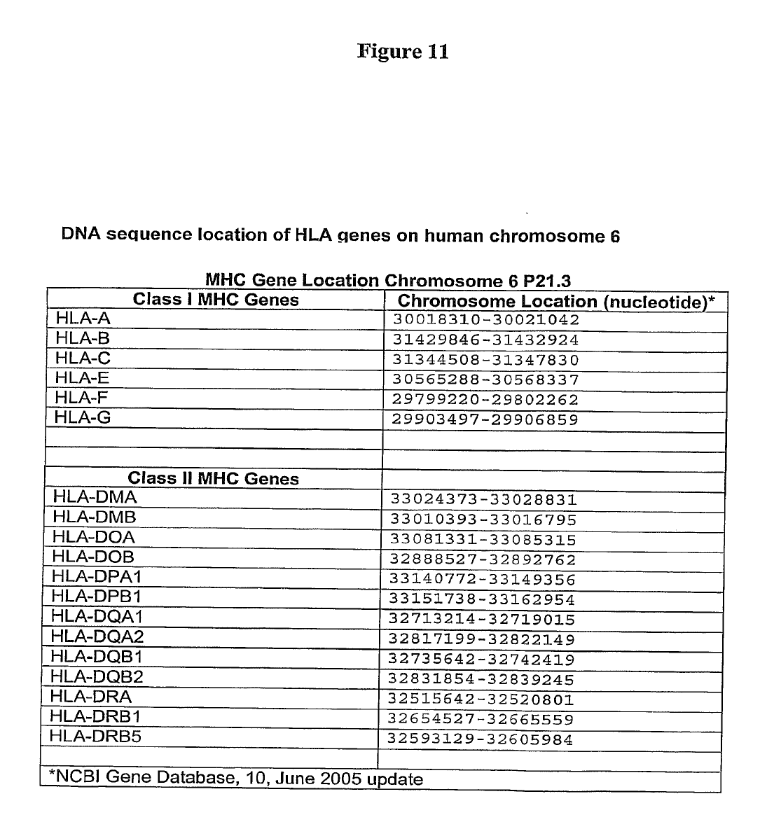

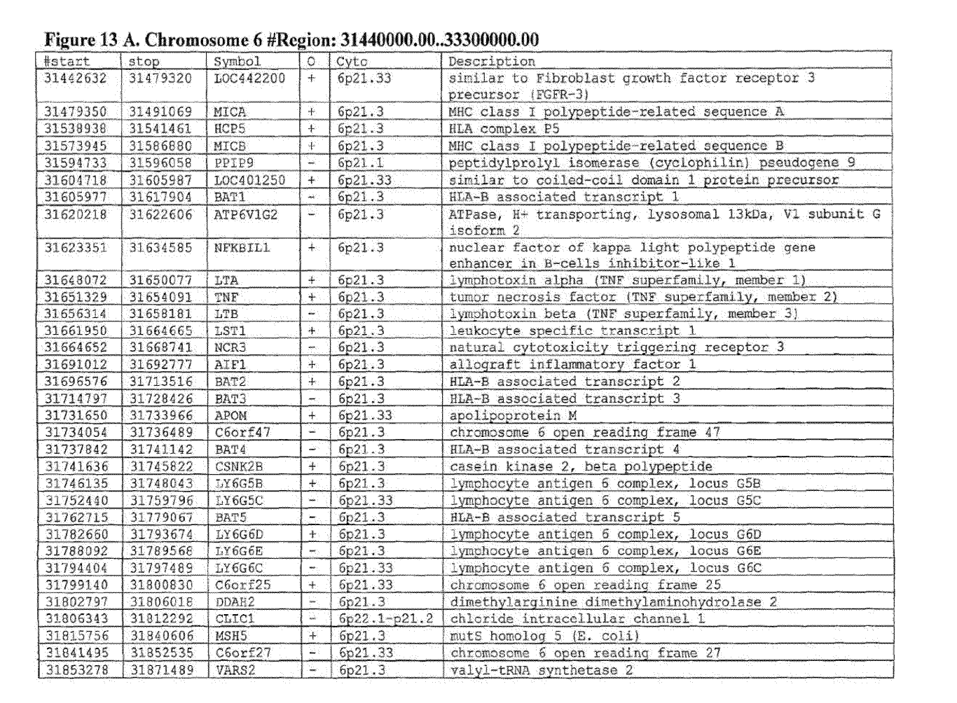

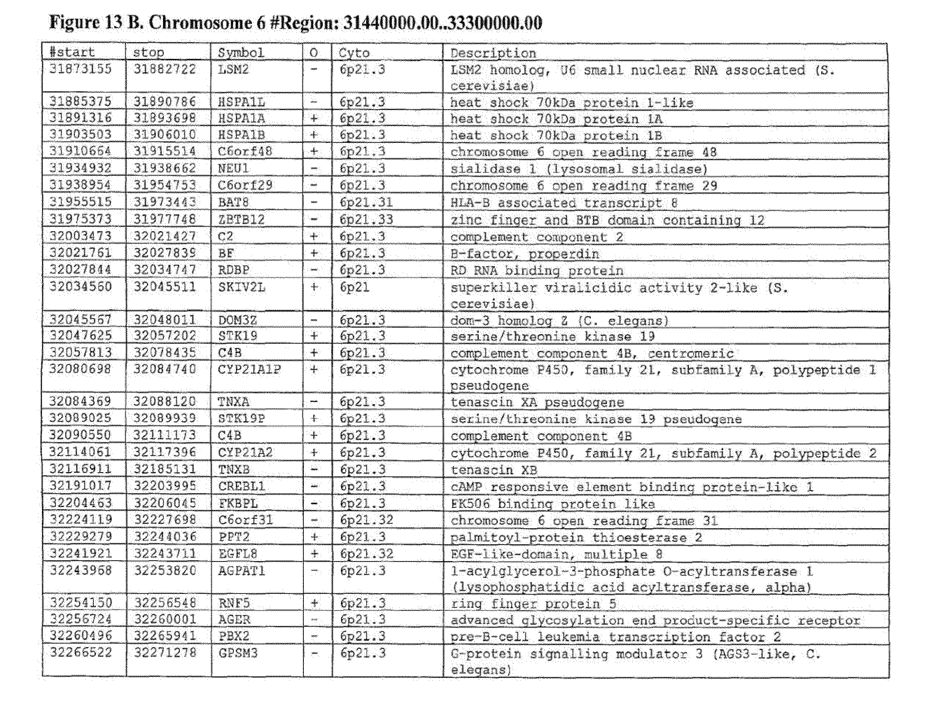

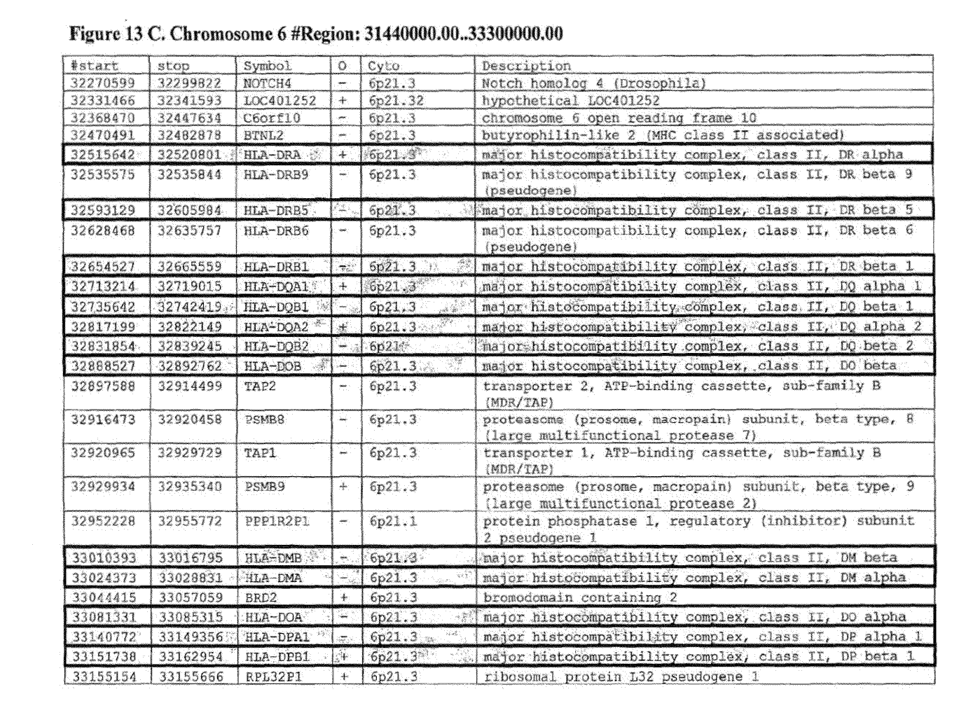

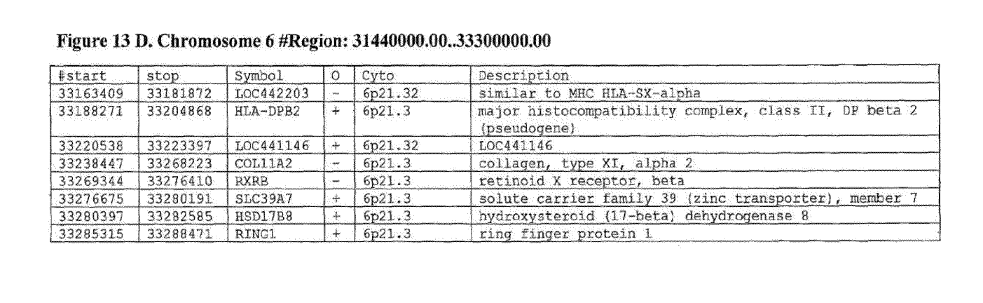

In another aspect, gene targeting is used to modify or delete HLA haplotypes in cells. Homologous recombination between a gene targeting vector that is homologous to a chromosomal gene introduces new genetic material to the chromosomal target (FIG. 2). In one embodiment, the invention provides a gene targeting vector for homologous recombination with the HLA region. The gene targeting vector may comprise one or more drug selectable markers (e.g., the Neomycin resistance gene or the Herpes simplex (HSV) virus Thymidine Kinase gene) and at least two kilobase pairs of DNA sequence homologous to a chromosomal target (e.g., one or more genes in the HLA region). For gene targeting of HLA genes, the gene targeting vector would include DNA sequence to one or more of the HLA gene sequences (FIGS. 11, 12, and 13). The gene targeting vectors of the invention may further comprise sequences for the Cre/LoxP and/or the FLP/FRT site specific recombinases. The gene targeting vectors of the invention may further comprise the sequence for the I-SceI rare cutting endonuclease. These DNA sequence elements may allow further chromosomal engineering to delete HLA genes and for site specific introduction of new HLA genes.

In one embodiment of the invention, a positive selection strategy is provided for selecting cells that are homozygous or hemizygous for desired gene structures. The positive selection strategy selects for cells expressing higher levels of the neomycin resistance gene by growing cells in higher levels of G418.

Positive selection of cells for gene duplications of the Neomycin resistance gene is experimentally straightforward. This selection strategy is designed to select for cells homozygous for HLA genes that have been modified by gene targeting to introduce the Neomycin resistance gene into defined chromosomal HLA genes. The concentration of G418 that is used for the purposes of the invention may be experimentally determined, but may range, for example, from about 0.001 mg/ml to about 100 mg/ml. Preferably, the concentration of G418 is about 0.01 mg/ml to about 25 mg/ml. More preferably, the concentration of G418 is about 0.1 mg/ml to about 10 mg/ml. To isolate cells homozygous for the targeted HLA gene, about 10⁵ to about 10⁹ cells are treated with G418. G418 resistant colonies are picked and expanded for storage. The G418 resistant colonies may be characterized by techniques sufficient to analyze the genotype of the cell, such as PCR or southern hybridization. Whether LOH is due to chromosome loss/duplication, interstitial deletion, or interchromosomal recombination may be determined by PCR of flanking chromosomal microsatellite sequences to identify the remaining haplotypes. Karyotyping may also be used to confirm chromosomal structure and number.

In another embodiment, a negative selection strategy is provided for selecting cells that are homozygous or hemizygous for desired gene structures. This negative, selection strategy involves selecting for cells that have lost the HSV TK gene by selecting for cell growth in the presence of Ganciclovir. This has particular application to selecting for cells expressing only one human HLA haplotype for creating hES cell banks with reduced HLA complexity. Negative selection of cells for loss of HSV TK in gene targeted HLA genes may be performed by growth in the presence of Ganciclovir and is experimentally similar to the G418 selections described above. To isolate cells missing HSV TK by LOH, about 10⁵ to about 10⁹ cells are treated with Ganciclovir. Characterization of the LOU products and chromosomes may utilize any of the characterization methods described above.

In a further aspect, cells that have lost specific HLA cell surface antigens may also he negatively selected by the use of complement mediated cell killing. Any hES cell line may be used Exemplary cell lines that are already typed for MHC loci are shown in FIG. 10. HLA alleles in new hES cell lines and GMP derived cell lines may be typed by PCR or serological assays. Antisera and complement for selection against specific HLA cell surface antigens may be purchased, for example, from DynalBiotech (Brown Deer, Wis.) or One Lambda (Canoga Park, Calif.).

The HLA complement mediated immunoselection approach is similar to that used for the isolation of HLA-A2 mutants from lymphoblastoid cell lines (Turner, et al., Proc Natl Acad Sci USA 85:3189-3192 (1988), de Nooij-van Dalen, et al., Mutat Res 374:51-62 (1997), de Nooij-van Dalen, et al., Genes Chromosomes Cancer 21:30-38 (1998), de Nooij-van Dalen, et al., Genes Chromosomes Cancer 30:323-335 (2001), de Nooij-van Dalen, et al., Mutat Res 423:1-10 (1999), Pious, et al., Proc Natl Acad Sci USA 70:1397-1400 (1973), Janatipour, et al., Mutat Res 198:221-226 (1988). Selections are performed by resuspension of 10⁶ cells in 100 μl of monoclonal antibody directed against one HLA allele, and incubating for 30 min at 4° C. After the addition of 5 ml medium, the cells are centrifuged, then resuspended in 200 ml of undiluted absorbed complement, and than are incubated for 45 minutes at 37° C., with continuous shaking. The cells are washed with 5 ml of medium and a second round of selection is performed by resuspending the cells in 200 μl of a mix of antibody/complement (75 μl/125 μl). After 30 minutes at 37° C., the cells are immediately diluted with culture medium to 5×10⁴ cells/ml and kept on ice until plating. After 2 weeks, a 10 μl cell suspension of each surviving clone are replica-plated into a 24-well plate and are subjected to reselection with 30 μl antibody/complement (10 μl/20 μl) for 30 min at room temperature, are followed by the addition of 160 μl medium. Surviving clones are scored after 3 days.

To avoid non-specific killing, complement is pre-absorbed to cells that will be used for LOU selection. Complement is slowly defrosted on ice and incubated twice with 10⁷ cells per ml on ice for 45 min, with continuous shaking. After centrifugation at 48° C., the supernatant is filtered (0.8 μM) and stored at −20° C.

LOU frequencies are influenced by proteins that mediate DNA stability and by DNA damaging agents. Loss of p53 results in higher LOU in mouse T lymphocytes and changes the mechanism of LOU from predominantly mitotic recombination events to LOU via interstitial deletion and chromosomal loss. In addition, treatment of mice with gamma radiation resulted in an increase in tissue specific LOU events. Treatment of cells with siRNA targeted to p53 induces transient downregulation of p53 protein sensitizing cells to LOU. A similar approach is to transiently transfect cells with expression vectors encoding the human pappiloma virus E6 protein or adenovirus FAB gene, both of which destabilize or inactivate p53. Treatment with small molecule drugs and vectors that interfere with other proteins involved with genomic stability or mitosis will likely provide alternative treatments to increase the frequency and spectrum of LOH events in somatic and stem cells. Some of these drugs would include the spindle poisons or antimitotic agents, okdaic acid, colchicine, vincristine, demecolcine, nocodazole, and colecimid.

Chromosomes can be engineered by gene targeting technologies in living cells, in permiabilized cells to be used for nuclear transfer, or in chromosomal masses in vitro to enable selection for LOH or to engineer LOH by physically manipulating or destroying target chromosomes. In certain embodiments, the chromosome carrying the MHC genes can be removed from cells by laser ablation and a chromosome carrying the identical chromosome as remains in the cell can be added by microsome-mediated chromosome transfer, or by other techniques known in the art. A cell's mitotic apparatus (e.g., spindles, kinetocores, etc.) may also be disrupted by laser. Engineered LOH may also be performed by optically trapping chromosomes in dividing cells to prevent segregation; in isolated nuclei by homologous recombination through treatment of permealized nuclei with nucleic acids and recombination proteins and selection in reconstituted cells using drug selectable markers or cell surface antigens as described herein; in chromatin masses by chromosomal laser ablation of specific chromosomes for use in nuclear transfer; in chromatin masses for use in nuclear transfer by laser tweezers to opto-mechanically remove specific chromosomes; or, in chromatin masses for use in nuclear transfer by atomic force microscopy to mechanically destroy specific chromosome integrity. Chromosomes may be morphologically identifiable or may be tagged with fluorescent labels such as, for example, triplex forming gene probes or probes coated with recombinases.

3. Generation of Cell Lines Homozygous or Hemizygous for MHC Antigens

Hemizygous or homozygous HLA cell lines may be generated in stem cell lines such as ES, EG, or ED cells from human or non-human animals, or may be generated in differentiated cell lines that are dedifferentiated to generate a totipotent or pluripotent stem cell line that is homozygous at the HLA locus. Methods for dedifferentiating cells are known in the art. See for example U.S. Patent Publication No. US 2004/0091936, filed May 14, 2004, the disclosure of which is incorporated by reference herein.

For instance, differentiated cells can be dedifferentiated using reprogramming methods to generate a totipotent or pluripotent stem cell. Totipotent and pluripotent stem cells homozygous for histocompatibility antigens, e.g., MHC antigens can be produced by transferring cytoplasm from an undifferentiated cell such as an oocyte or an ES cell into a somatic cell that is homozygous for MHC antigens, so that the chromatin of the somatic cell is reprogrammed and the somatic cell de-differentiates to generate a pluripotent or totipotent stem cell. Cytoplasm from an undifferentiated cell may also be added to isolated nuclei or chromatin from undifferentiated cells, or undifferentiated cells that are permeabilized. Methods for converting differentiated cells into de-differentiated, pluripotent, stem or stem-like cells that can be induced to re-differentiate into a cell type other than that of the initial differentiated cells, are described, for example, in U.S. application Ser. No. 09/736,268, filed Dec. 15, 2000, and U.S. application Ser. No. 10/112,939 filed Apr. 2, 2002, the disclosures of both of which are incorporated herein by reference in their entirety.

In the first step, designated the nuclear remodeling step, the degree of reprogramming of the somatic cell genome is increased and the problem of access to oocytes of the same species as the somatic cell is alleviated by the use of any or a combination of several novel reprogramming procedures. In all of these procedures, the somatic cell nucleus is remodeled to replace the components of the nuclear envelope with those of an undifferentiated cell. Simultaneously, or at a point in time soon enough to prevent the inclusion of somatic cell differentiated components incorporating within the nuclear envelope, the chromatin of said cell is reprogrammed to express genes of an undifferentiated cell.

In the second step, designated herein as the cellular reconstitution step, the nucleus, containing the remodeled nuclear envelope of step one is fused with a cytoplasmic bleb containing requisite mitotic apparatus, and capable, together with the transferred nucleus, of producing a population of undifferentiated stem cells such as ES or ED-like cells capable of proliferation.

In the third step, colonies of cells arising from one or a number of cells resulting from step two are characterized for the extent of reprogramming and for the normality of the karyotype and colonies of a high quality are selected. While this third step is not required to successfully reprogram cells and is not necessary in some applications, such as in analyzing the molecular mechanisms of reprogramming, for many uses, such as when reprogramming cells for use in human transplantation, the inclusion of the third quality control step is preferred. Colonies of reprogrammed cells that have a normal karyotype but not a sufficient degree of reprogramming may be recycled by repeating steps 1-2 or 1-3.

The nucleus being remodeled in step one may also be modified by the addition of extracts from cells such as DT40 known to have a high level of homologous recombination. The addition of DNA targeting constructs with the DNA and the extracts from cells permissive for a high level of homologous recombination will then yield cells after reconstitution in step 2 and screening in step 3 that have a desired genetic modification.

4. Modified Stem Cell Lines

The methods of the present invention include producing totipotent and/or pluripotent stem cells homozygous for MHC antigens that are genetically modified relative to the cells of the human donor from which they were originally obtained. The stem cells can be genetically modified in any manner that enhances or improves the overall efficiency by which cells for transplant are produced and the therapeutic efficacy of the cell transplantation. Methods that use recombinant DNA techniques to introduce modifications at selected sites in the genomic DNA of cultured cells are well known. Such methods can include (1) inserting a DNA sequence from another organism (human or non-human) into target nuclear DNA, (2) deleting one or more DNA sequences from target nuclear DNA, and (3) introducing one or more base mutations (e.g., site-directed mutations) into target nuclear DNA. Such methods are described, for example, in Molecular Cloning, a Laboratory Manual, 2nd Ed., 1989, Sambrook, Fritsch, and Maniatis, Cold Spring Harbor Laboratory Press; U.S. Pat. No. 5,633,067, “Method of Producing a Transgenic Bovine or Transgenic Bovine Embryo, DeBoer et al., issued May 27, 1997; U.S. Pat. No. 5,612,205, “Homologous Recombination in Mammalian Cells,” Kay at al., issued Mar. 18, 1997; and PCT publication NO 93/22432, “Method for Identifying Transgenic Pre-implantation Embryos,” all of which are incorporated by reference herein in their entirety. Such methods include techniques for transfecting cells with foreign DNA fragments and the proper design of the foreign DNA fragments such that they effect insertion, deletion, and/or mutation of the target DNA genome. For example, known methods for genetically altering cells that use homologous recombination can be used to insert, delete, or rearrange DNA sequences in the genome of a cell of the present invention. A genetic system that uses homologous recombination to modify targeted DNA sequences in a mammalian cell to “knock-out” a cell's ability to express a selected gene is disclosed by Capecchi et al. in U.S. Pat. Nos. 5,631,153 and 5,464,764, the contents of which are incorporated herein in their entirety. Such known methods can be used to insert into the genomic DNA of a cell an additional (exogenous) DNA sequence comprising an expression construct containing a gene that is to be expressed in the modified cell. The gene to be expressed can be operably linked to any of a wide variety of different types of transcriptional regulatory sequences that regulate expression of the gene in the modified cell. For example, the gene can be under control of a promoter that is constitutively active in many different cell types, or one that is developmentally regulated and is only active in one or a few specific cell types. Alternatively, the gene can be operably linked to an inducible promoter that can be activated by exposure of the cell to a physical (e.g., cold, heat, light, radiation) or chemical signal. Many such inducible promoters and methods for using them effectively are well known. Examples of the characteristics and use of such promoters, and of other well-known transcriptional regulatory elements such as enhancers, insulators, and repressors, are described, for example, in Transgenic Animals, Generation and Use, 1997, edited by L. M. Houdebine, Hardwood Academic Publishers, Australia, the contents of which are incorporated herein by reference.

Stem cells homozygous for MHC antigens that have multiple genetic alterations can be produced using known methods. For example, one can produce cells that are modified at multiple loci, or cells that are modified at a single locus by complex genetic alterations requiring multiple manipulations. To produce stem cells having multiple genetic alterations, it is useful to perform the genetic manipulations on somatic cells cultured in vitro, and then to clone the genetically altered cells by somatic cell nuclear transfer and generate ES cells having multiple genetic alterations from the resulting blastocysts. Methods for generating genetically modified cells using nuclear transfer cloning techniques are described, for example, in U.S. application Ser. Nos. 09/527,026 filed Mar. 16, 2000, 09/520,879 filed Apr. 5, 2000, and 09/656,173 filed Sep. 6, 2000, the disclosures of which have been incorporated herein by reference in their entirety.

Alternatively, the totipotent and/or pluripotent stem cells having homozygous MHC alleles that are produced by any of the methods described above can be genetically modified directly using known methods. For example, Zwaka et al. have described a method for genetically modifying human ES cells in vitro by homologous recombination (Nature Biotechnology 21319-321 (2003)).

In generating stem cells by nuclear transfer, it is useful to genetically modify the nuclear donor cell to enhance the efficiency of embryonic development and the generation of ES cells. The gene products of the Ped type, which are members of the Class I MHC family and include the Q7 and Q9 genes, are reported to enhance the rate of embryonic development. Modification of the DNA of nuclear donor cells by insertion of DNA expression constructs that provide for the expression of these genes, or their human counterparts, will give rise to nuclear transfer embryos that grow more quickly. It appears that these genes are only expressed early in blastocyst development and so are not expected to be disruptive of later development.

The efficiency of embryonic development can also be enhanced by genetically modifying the nuclear donor cell to have increased resistance to apoptosis. Genes that induce apoptosis are reportedly expressed in preimplantation stage embryos (Adams et al., Science, 281(5381):1322-1326 (1998). Such genes include Bad, Bok, BH3, Sik, Hrk, BNIP3, Bim.sub.L, Bad, Bid, and EGL-1. By contrast, genes that reportedly protect cells from programmed cell death include BcL-XL, Bcl-w, Mcl-1, Al, Nr-13, BHRF-1, LMW5-HL, ORF16, Ks-Bel-2, E1B-19K, and CED-9. Nuclear donor cells can be constructed in which genes that induce apoptosis are “knocked out” or in which the expression of genes that protect the cells from apoptosis is enhanced or turned on during embryonic development. Expression constructs that direct synthesis of antisense RNAs or ribozymes that specifically inhibit expression of genes that induce apoptosis during early embryonic development can also be inserted into the DNA of nuclear donor cells to enhance development of nuclear transfer-derived embryos. Apoptosis genes that may be expressed in the antisense orientation include BAX, Apaf-1, and caspases. Many DNAs that promote or inhibit apoptosis have been reported and are the subject of numerous patents. The construction and selection of genes that affect apoptosis, and of cell lines that express such genes, is disclosed in U.S. Pat. No. 5,646,008, the contents of which are incorporated herein by reference.

Stem cells could be genetically modified to grow more efficiently in tissue culture than unmodified cells. This could be accomplished by, for example, increasing the number of growth factor receptors on the cells' surface. Use of stem cells having such modifications reduces the time required to generate an amount of cells for transplant that is sufficient to have therapeutic effect.

The histocompatibility of a line of cells to be used for transplant with a transplant recipient may be increased by altering the genomic DNA of the cells to replace a non-homozygous MHC allele with one that is homozygous and matches an HLA allele of the recipient patient. Alternatively, the genomic DNA of the cells can be modified to inhibit the effective presentation of a class I or class II HLA antigen on the cell's surface; by, for example, introducing a genetic alteration that prevents expression, of .beta.2-microglobulin, which is an essential component of class I HLA antigens; by introducing genetic alterations in the promoter regions of the class I and/or or class II MHC genes; or simply by deleting a portion of the DNA of one or more of the class I and/or or class II MHC genes sufficient to prevent expression of the gene(s).

Stem cells of the invention can be genetically modified (e.g., by homologous recombination) to have a heterozygous knock-out of the Id1 gene, and a homozygous knockout of the Id3 gene. As described in PCT Application No. PCT/US03/01827 (WO 03061591, published Jul. 31, 2003, herein incorporated by reference in its entirety) (Stem Cell-Derived Endothelial Cells Modified to Disrupt Tumor Angiogenesis), filed Jan. 22, 2003, these stem cells can be induced to differentiate into Id1.+−., Id3−/− endothelial cell precursor cells that are useful for the treatment of cancer because they give rise to endothelial cells that disrupt and inhibit tumor angiogenesis.

Stem cells of the invention can also be genetically modified to provide a therapeutic gene product that the patient requires, e.g., due to an inborn error of metabolism. Many genetic diseases are known to result from an inability of a patients cells to produce a specific gene product. The present invention provides genetically altered stem cells that can be used to produce cells with homozygous MHC alleles for transplantation, cells that are genetically modified to synthesize enhanced amounts of a gene product required by the transplant recipient. For example, hematopoietic stem cells that are genetically altered to produce and secrete adenosine deaminase can be prepared for transplant to a patient suffering from adenosine deaminase deficiency. The methods of the present invention permit production of such cells without the use of recombinant retrovirus, which can insert at a site in the genomic DNA that disrupts normal growth control and causes neoplastic transformation.

Stem cells of the invention can also be genetically modified by introduction of a gene that causes the cell to die, such as with a suicide gene. The gene could be put under control of in inducible promoter. If for any reason the transplanted cells react in a way that can harm the recipient, induction of the expression of the suicide genes kills the transplanted cells. Use, of inducible suicide genes in this manner is known in the art. Suitable suicide genes include, for example, genes encoding HSV thymidine kinase and cytodine deaminase, with which cell death is induced by gancyclovir and 5-fluorocytosine, respectively.

The cells may be modified to knockout one or more histocompatibility antigen alleles, e.g., MHC alleles such that only one set remains. This leads to an underexpression of the MHC genes, but a phenotype effective in reducing the complexity of the MHC serotype and effective in producing cells capable of otherwise functioning and useful in the treatment of disease. Alternatively, homozygosity can be engineered into the cell lines by the targeted introduction of the appropriate alleles to the nonhomologous set, to result in homozygosity.

### Applications

The invention provides methods and compositions that are generally useful in the treatment of disease by providing cells for use in mammalian and human cell therapy. The invention also provides methods and compositions useful in medical and biological research. For example, the cells with reduced complexity in the HLA genes are useful, such as human cells useful in treating dermatological, dental, respiratory, ophthalmological, cardiovascular, neurological, endocrinological, skeletal, and blood cell disorders. The cells and banks of this invention are also useful in any grafts.

In certain embodiments of the invention, cells with reduced complexity in the HLA genes are utilized in in research and/or the treatment of disorders relating to cell biology, drug discovery, and in cell therapy, including but not limited to production of hematopoietic and hemangioblastic cells for the treatment of blood disorders, vascular disorders, heart disease, cancer (e.g., tumor angiogenesis), and wound healing, pancreatic beta cells useful in the treatment of diabetes, retinal cells such as neural cells and retinal pigment epithelial cells useful in the treatment of retinal disease such as retinitis pigmentosa and macular degeneration, neurons useful in treating Parkinson's disease, Alzheimer's disease, chronic pain, stroke, psychiatric disorders, and spinal cord injury, cardiac muscle cells useful in treating heart disorders such as heart failure or infarction, skin cells useful in treating wounds for scarless wound repair, burns, promoting wound repair, and in treating skin aging, liver cells for the treatment of liver disease such as cirrhotic liver disease, kidney cells for the treatment of kidney disease such as renal failure, cartilage for the treatment of arthritis, lung cells for the treatment of lung disease, muscle cells for the treatment of age-related muscle atrophy and muscular dystrophy and bone cells useful in the treatment of bone disorders such as osteoporosis.

The disclosures of all references, patents and publications cited herein are hereby incorporated by reference.

The following examples are chosen to illustrate the methods for engineering the HLA genes in hES cells. While in example 1, the gene modification and homogenization of the modified HLA-A allele by LOH are described, the same strategy can be used to modify other HLA alleles, as can the approaches described in examples 2-9. The present invention is by no means limited to the following examples.

### EXAMPLES

### Example 1

### Engineering hES Cells for Homozygosity at the HLA-A Gene

Step 1: Gene Knockout of the HLA-A*010101 Allele

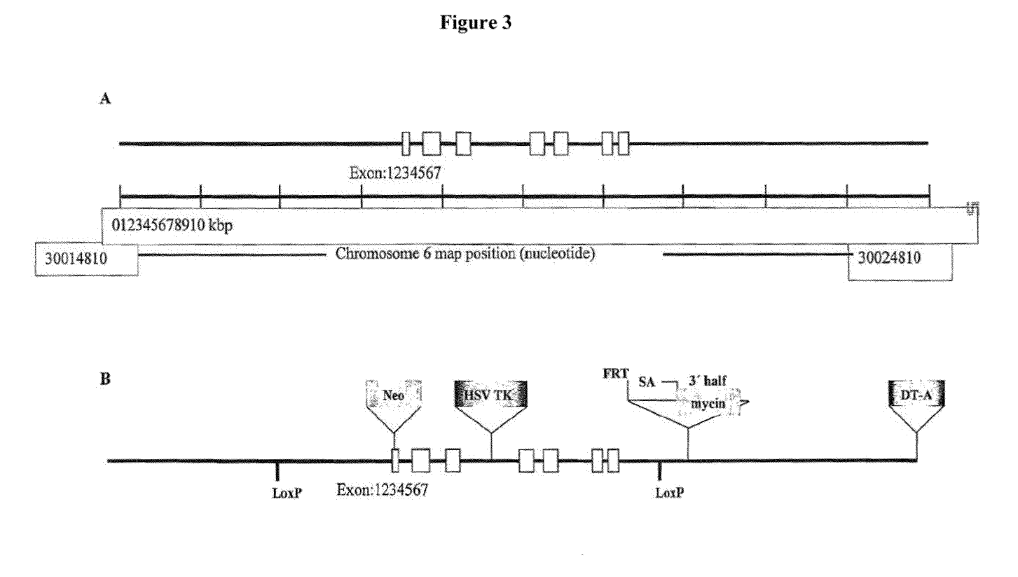

Female human embryonic stern cells generated under UMP conditions under pathogen-free conditions with an O-ABO blood type (hES (O−)) are modified using a replacement type gene targeting vector similar in structure to that diagrammed in FIGS. 2 and 3. In this approach, homologous recombination between the targeting vector and its homologous chromosomal gene target introduces selectable gene markers and other gene changes into the target site. Other gene changes can include point mutations, insertions, and deletions that may inactivate or change the function of the target gene. The neomycin acetyl transferase gene that confers cell resistance to the drug G418 included as a positive selectable marker to select for potential homologous recombinants. Other positive selectable markers can be gene expression cassettes that include genes encoding hygromycin phosphotransferase, puromycin acetyltransferase, blasticidin deaminase, guanine phosphoribosyltransferase, hypoxanthine/guanine phosphoribosyltransferase, adenine phosphoribosyltransferase, dihydrofolate reductase, and thymidine kinase. Other selectable makers that would allow positive screening or enrichment for recombinant cells by fluorescence activated cell sorting (FACS) include green fluorescent protein (and its derivatives), beta galactosidase, and cell surface antigens. A negative selectable marker is included at the linearized ends of the targeting vector that is deleted on recombination and can also be used to select for potential homologous recombinants. Other negative selectable markers that can be used are gene expression cassettes that include genes encoding guanine phosphoribosyltransferase, hypoxanthine/guanine phosphoribosyl transferase, adenine phosphoribosyltransferase, thymidine kinase, nitroreductase, ricin toxin, and diphtheria toxin A chain. The negative selectable HSV TK gene cassette is included in this targeting vector as an alternative negative selectable marker that is used to select for cells deleted for the HLA-A*010101 allele by treatment with the Cre recombinase.

The human HLA-A gene is located on chromosome 6p21.3 and its gene contains 8 exons, with the HLA-A peptide encoded in exons 1 through 7. Exon 1 encodes the leader peptide, exons 2 and 3 encode the alpha1 and alpha2 domains, exon 4 encodes the alpha3 domain, exon 5 encodes the transmembrane region, and exons 6 and 7 encode the cytoplasmic tail. Polymorphisms within exon 2 and axon 3 are responsible for the peptide binding specificity of each class one molecule. There are approximately 371 alleles of HLA-A that have been identified as of April 2005 (http://www.anthonynolan.org.uk/HIG/lists/classllist.html). While gene modification for the HLA-A allele 010101 is described, genetic modification for any other class I or class II HLA alleles is done by an identical process.

The HLA-A gene targeting vector is diagrammed in FIG. 3. Isogenic homologous HLA-A*010101 DNA for the targeting vector is obtained by long distance PCP, and subcloned into the blusescript vector pSK. The drug selectable markers, and “socket” cassette, are inserted into the targeting vector DNA using conventional recombinant DNA methods. A positively selectable neomycin expression cassette is cloned into exon 1 between nucleotides 3502 and 3503. A negatively selectable Herpes simplex virus thymidine kinase gene is cloned into intron 3 between nucleotides 5010 and 5011. A “socket” cassette containing a FRT, FLP recombinase recognition target sequence, heterologous intron, splice acceptor site and the 3′ half of a puromycin acetyl transferase gene expression cassette is cloned between nucleotides 7133 and 7134. The negatively selectable DT-A (diphtheria toxin chain A) gene expression cassette is cloned at the junction of the chromosome 6 DNA sequence and the vector backbone. The Cre recombinase LoxP recognition sequence is cloned between nucleotides 2010 and 2011, and 6811 and 6812, respectively.

The neomycin expression cassette allows for positive selection of homologous recombinant cells and cells with randomly integrated vector by growth in the presence of the drug G418. In homologous recombinants, the Neo cassette interrupts the HLA-A open reading frame leading to loss of HLA expression. Homologous recombinant cells in HLA-A can be doubly selected by simultaneously growing cells in G418 and by treatment with antibody to HLA-A*010101 and complement mediated cell killing. The DT-A gene allows for further enrichment of homologous recombinants since only cells that have lost the DT-A gene through homologous recombination, or have inadvertently lost DT-A gene expression by mutation, will survive.

The LoxP and FRT recombinase recognition sequences allow recombinase mediated gene modifications of homologous recombinant cells. The LoxP sequences permits high frequency deletion of intervening HLA-A*010101 gene sequences for complete deletion of the allele and deletion of the Neo and HSV TK expression cassettes. Cells deleted for the HLA-A*010101 allele by recombination between the LoxP recognition sequences will have lost the HSV TK gene and are selected by growth in the drug Ganciclovir. Ore recombinase has been used to efficiently delete hundreds of basepairs to megabasepairs of DNA in mammalian cells. The FRT “socket” cassette allows for positive selection of FLP recombinase mediated gene insertions into HLA-A locus genomic DNA sequences. Only FLP recombinase mediated events that reconstruct a functioning puromycin acetyltransferase gene will grow in the presence of the drug puromycin. Equivalent functional “socket” cassettes can be constructed out of the positive selectable and FACS markers described above.

To genetically modify the HLA-A*010101 allele by gene targeting, targeting vector, linearized on the 3′ side of the DT-A gene cassette, is electroporated into human embryonic stem cells (Zwaka and Thomson, Nat Biotechnol 21:319-321 (2003)). One week before electroporation, cells are plated onto Matrigel (Becton Dickinson, San Jose, Calif.) and cultured with fibroblast-conditioned medium. To remove colonies as intact clumps, cells are treated with trypsin (Klimanskaya and McMahon, Handbook of Stem Cells 1:437-450 (2004)), washed with medium, and resuspended in 0.5 ml of culture medium at a final titer of 3-6×10⁷ cells/ml. Five to ten minutes before electroporation, 10 to 40 μg of linearized targeting vector in phosphate buffered saline or in medium is added to the resuspended cells. Cells are added to a 0.4 cm electroporation cuvette and electroporated with a single 320 v, 200 μf pulse at room temperature using a Biorad Gene Pulsar II electroporator. Electroporated cells are incubated for 10 minutes at room temperature and plated onto a 10 cm Petri dish coated with Matrigel. G418 is added to a final concentration of 50 to 200 μg/ml 48 hours post electroporation. G418 resistant colonies are picked after approximately 3 weeks and analyzed by PCR using primers specific for the Neo, HSV TK, and socket cassette and by PCR from the “socket” cassette and flanking genomic sequence. Colonies positive for gene targeting identified by PCR are confirmed by southern hybridization.

Step 2a: Engineering cells homozygous for the HLA-A*010101 gene knockout using complement mediated cytotoxicity to select for cells with LOH selection using HLA-A010101 specific antibody and complement mediated cytoxicity (CMC) are performed by resuspending G418 resistant cells in 100 μl of monoclonal antibody directed against the HLA-A allele present on untargeted sister chromosome, and incubated for 30 minutes at 4° C. After the addition of 5 ml medium, the cells are centrifuged, resuspended in 200 μl of undiluted absorbed complement, and incubated for 45 minutes (“min”) at 37° C. with continuous shaking. The cells are washed with 5 ml of medium and a second round of selection is performed by resuspending the cells in 200 μl of a mix of antibody/complement (75 μl/125 μl). After 30 minutes at 37° C., the cells are immediately diluted with culture medium and kept on ice until plating. Two to three weeks later, the plates are scored, and clones from the selection plates are retreated with 30 μl antibody/complement (10 μl/20 μl) for 30 minutes at 37° C. to eliminate contaminating wild type clones.

To avoid non-specific killing, complement is pre-absorbed to cells that are used for LOH selection. Complement is slowly defrosted on ice and incubated twice with 1×10⁷ cells per ml on ice for 45 minutes, with continuous shaking. After centrifugation at 4° C. the supernatant is filtered and stored at −20° C.

The gene structure of G418r, CMC-surviving clones are analyzed by PCR and southern hybridization to confirm that the isolated cell clones are homozygous for the HLA-A*010101 gene knockout. Other class I and class II HLA loci are typed by PCR and serological testing to confirm the cellular HLA genotype. LOH by chromosome loss and reduplication or by homologous recombination will produce cell clones homozygous for all class I and class II HLA alleles.

Step 2b: Alternative selection for cells homozygous for the HLA-A*010101 gene knockout using drug resistance to select for cells with LOH

An alternative method that may be used to select for cells homozygous for the gene targeted HLA-A*010101 allele is by cell growth in high concentrations of G418 (for knockouts using the Neo gene). The objective of this approach is to select for cells with increased expression of the Neo gene drug resistance cassette by LOH through chromosome loss and duplication or by homologous recombination between homologous sister chromosomes. Both mechanisms generate a second copy of the Neo expression cassette and higher levels of neomycin actelytransferase expression. Selection for LOH by increased drug resistance can also be accomplished using other positive selectable drug markers described above.

Before selection, cells are plated onto a 10 cm Petri dish coated with Matrigel. G418 is added to a final concentration of 500 μg/ml to 2000 μg/ml. Two to three weeks later, surviving colonies are isolated, grown and analyzed by PCR and southern hybridization to confirm that the isolated cell clones are homozygous for the HLA-A*010101 gene knockout. Other class I and class II HLA loci are typed by PCR and serological testing to confirm the cellular HLA genotype. LOH by chromosome loss/duplication or by homologous recombination will produce cell clones homozygous for the HLA-A*010101 gene knockout and clones homozygous for other class I and class II HLA alleles.

### Example 2

### Inactivation of Both Cellular HLA-A Alleles Using Gene Targeting

Gene targeting may also be used to inactivate both sister copies of HLA-A. There are two gene targeting strategies used to generate sister knockouts, starting with the HLA-A knockout cell line illustrated in FIG. 3. One strategy is to construct a new gene targeting vector, replacing the Neo cassette with a new positive selection cassette, allowing positive drug selection for new homologous recombinants at the unmodified sister HLA-A allele. Co-selection of cells using both positive selectable markers ensures recovery of cells with both HLA-A alleles targeted. An alternative approach is to “recycle” the Neo drug resistance cassette, deleting the cassette by Cre mediated site specific recombination. To accomplish this, cells are transiently transfected with the Cre recombinase expression vector, and 5 to 7 days later put under selection with the drug Ganciclovir to select for cells missing the HSV TK gene. Cells deleted for Neo, HSV TK, and not expressing the targeted HLA-A*010101 allele are used for a second round of gene targeting using the original targeting vector to knockout the sister allele.

### Example 3

### Deletion of HLA-C and HLA-B Using a Gapped Replacement Targeting Vector

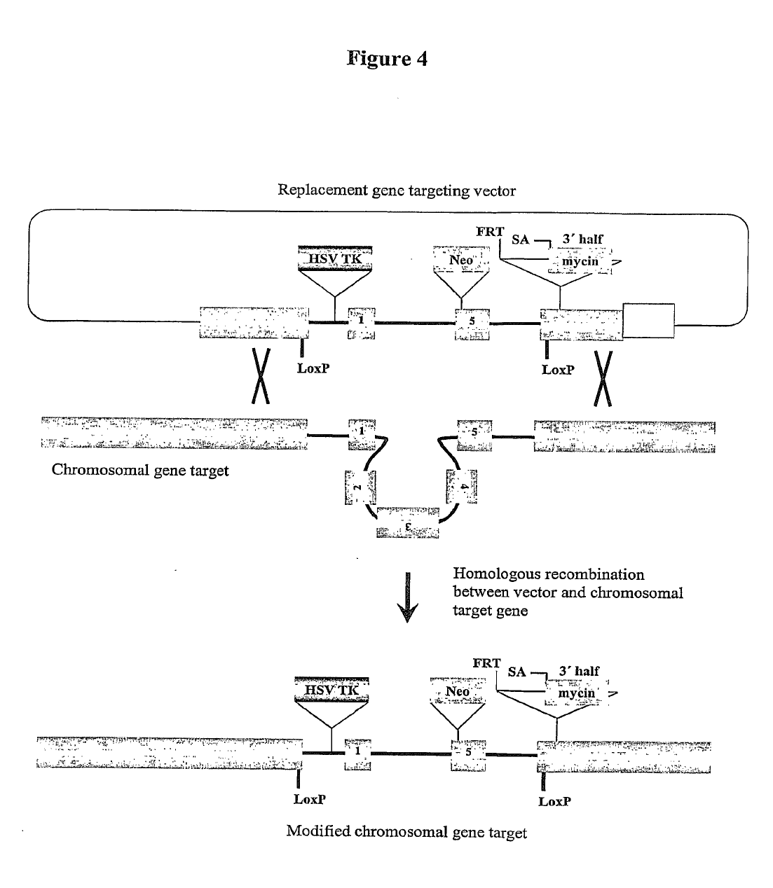

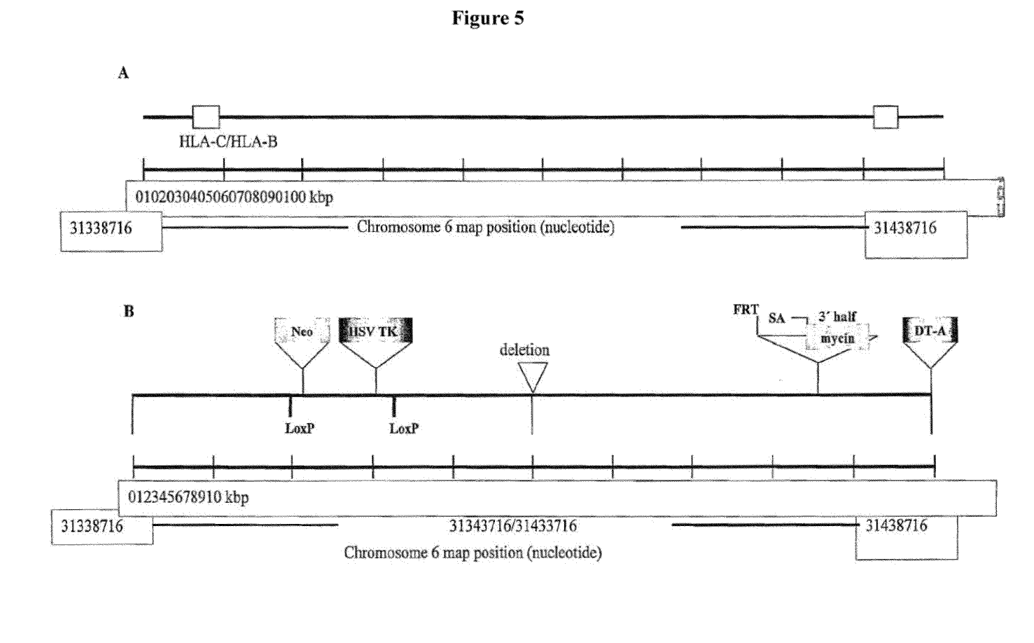

While the objective of many gene targeting strategies is to modify one gene, gene targeting vectors are used to delete from a few basepairs to several kilobasepairs of chromosomal target genes. The approach is graphically illustrated in FIGS. 4 and 5. Essentially a conventional replacement style vector is used, although defined chromosomal target DNA sequences are deleted from the vector. A successful targeted gene modification produces cells with the corresponding deleted chromosomal sequences.

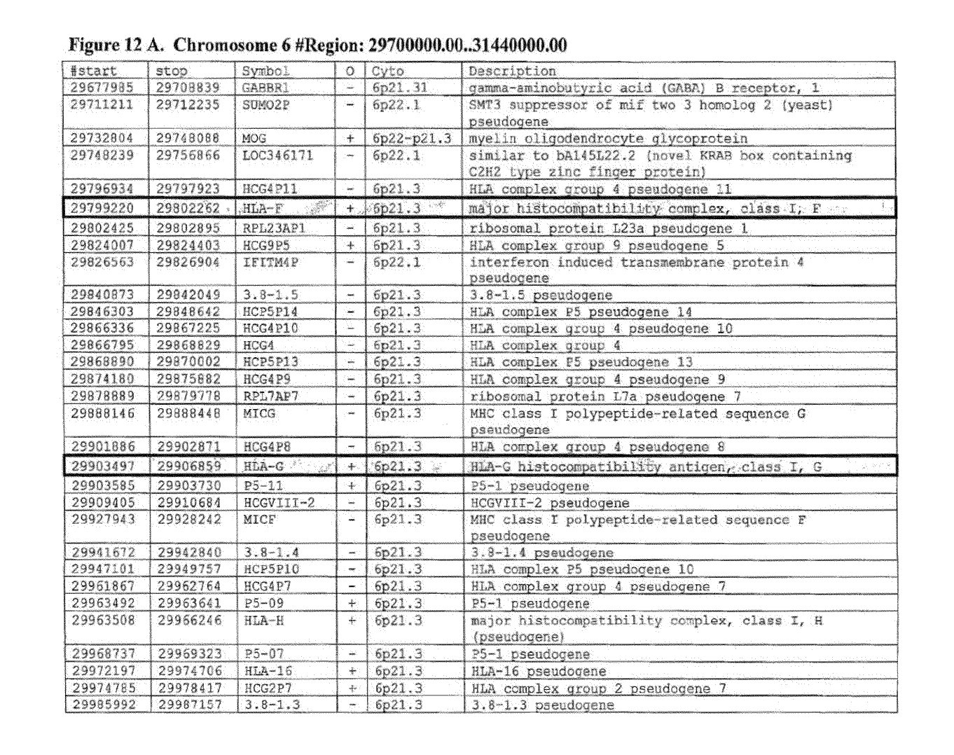

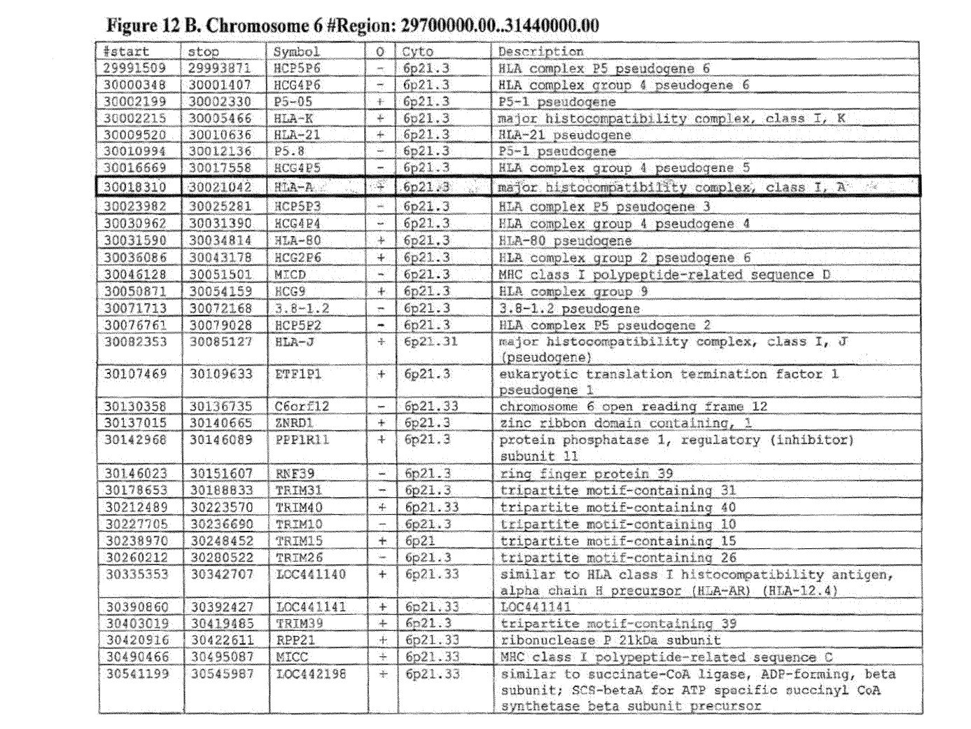

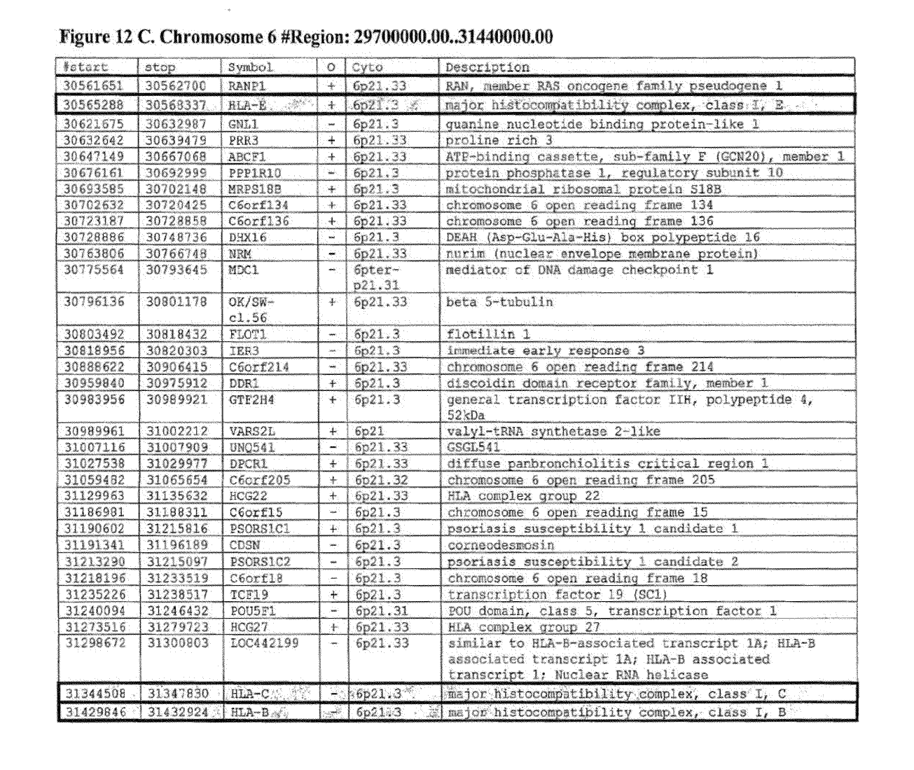

The HLA-C/HLA-B locus is illustrated in FIG. 5. The HLA-C and HLA-B structural genes are 4 to 5 kilobasepairs in size, separated by approximately 80 kilobasepairs of chromosomal DNA sequence. The sequence identities of HLA-C and HLA-B are defined in FIGS. 11 and 12. The chromosomal HLA-C and HLA-B genes are deleted using the targeting vector depicted in FIG. 5. In this approach, the targeting vector is missing 90 kilobasepairs of chromosomal sequences between nucleotide 31343715 and 31433716, deleting both HLA-C and HLA-B. There are 5 kilobasepair arms homologous to the chromosomal target sequences flanking the HLA-C and HLA-B genes for homologous recombination. The drug selectable markers and site specific recombinase recognition sequences are described above.

Gene targeting with the deletion vector is essentially identical to the protocol described above. Linearized targeting vector is electroporated into cells and potential homologous recombinants are selected with the drug G418. Enrichment for homologous recombinant cells may also be accomplished by CMC using HLA-C and HLA-B allele specific antibodies. Homologous recombinant cell lines are screened by PCR, southern hybridization, and serological methods to confirm the genetically modified gene structure and loss of HLA-C and HLA-B proteins.

Cell lines homozygous for the HLA-C/HLA-B deletion are generated by LOH and selected by CMC killing using antisera against the remaining HLA-C and HLA-B allele.

### Example 4

### Deletion of HLA-F, HLA-G, and HLA-A Genes by Site Specific Recombination

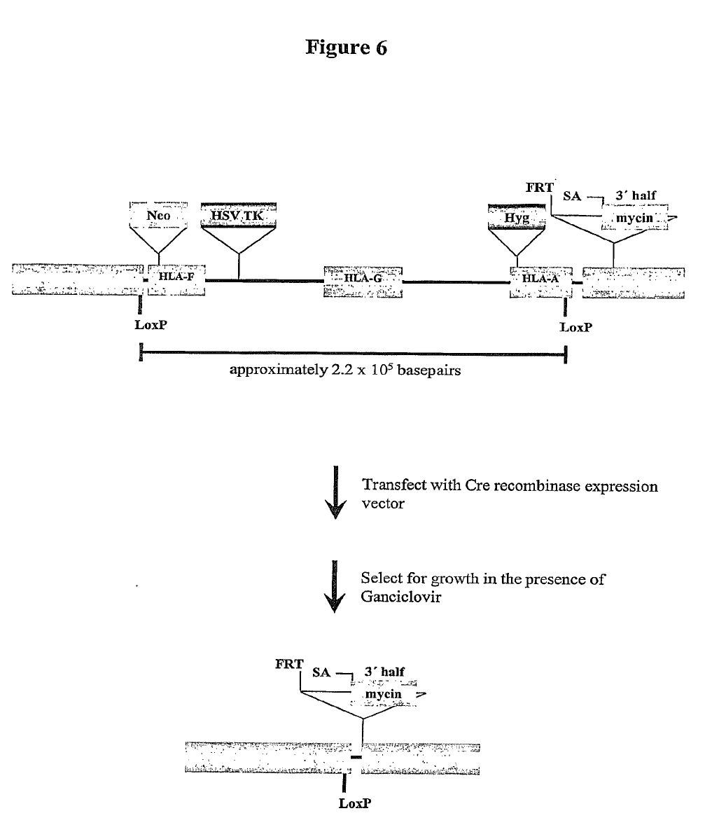

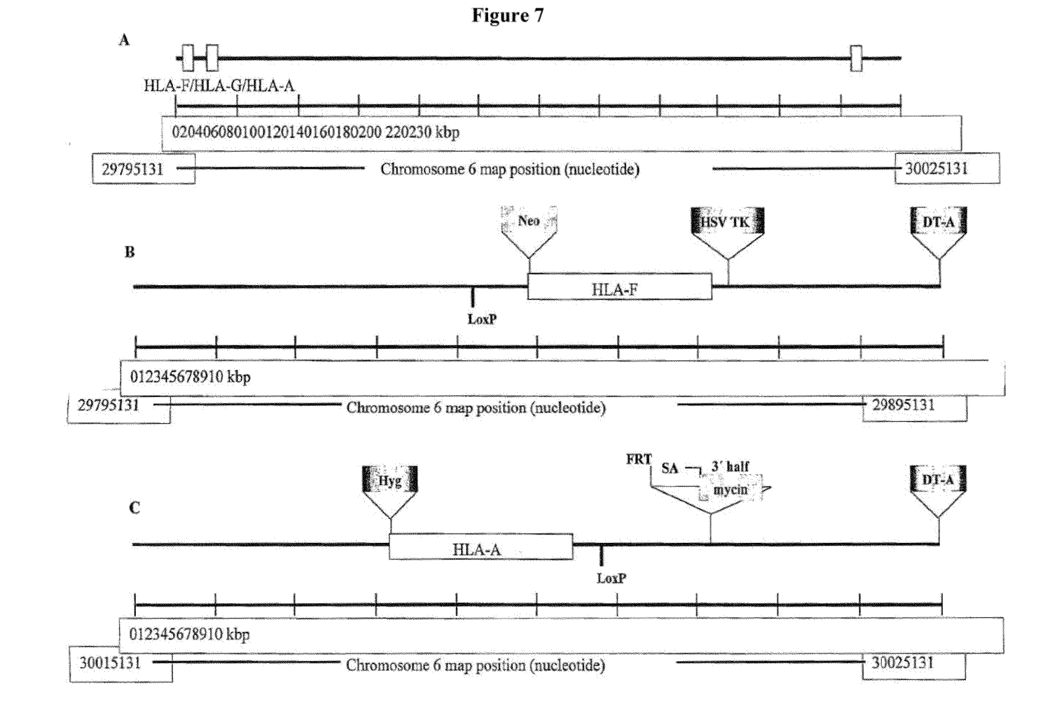

While gapped replacement vectors have no: been used to engineer large chromosomal deletions, site specific recombination between LoxP and FRT recognition sequences have been used to engineer deletions encompassing megabasepairs of chromosomal DNA. This approach requires two gene targeting steps to introduce LoxP or FRT sequences into their chromosomal targets (FIG. 6). The HLA-F/HLA-A locus and targeting vectors are diagrammed in FIG. 7. Once two tandemly oriented LoxP/FRT sequences have been targeted to the chromosome, site specific recombination catalyzes high frequency deletion between the recombinase recognition sequences (FIG. 6). This is accomplished by transient transfection of Cre or FLP recombinase expression cassettes into the gene targeted cell lines followed by selection for or against markers in the targeted genes. In this example, the HSV TK gene is present in the gene targeted HLA-F gene. Loss of HSV TK from site specific recombination allows cell growth in the presence of the drug Ganciclovir. Cells deleted for the HLA-G allele will also survive CMC killing with antisera to the HLA-G allele. In this approach, recombination between the LoxP sequences will leave behind a “socket” cassette for site specific recombination to introduce desired HLA genes to tailor cells for organ or tissue transplantation.

Cell lines homozygous for the HLA-F/HLA-A deletion are generated by LOH and selected by CMC killing using antisera against the remaining HLA-F, HLA-G, and HLA-A alleles.

### Example 5

### Reconstruction of HLA Expression by Site Specific Recombination

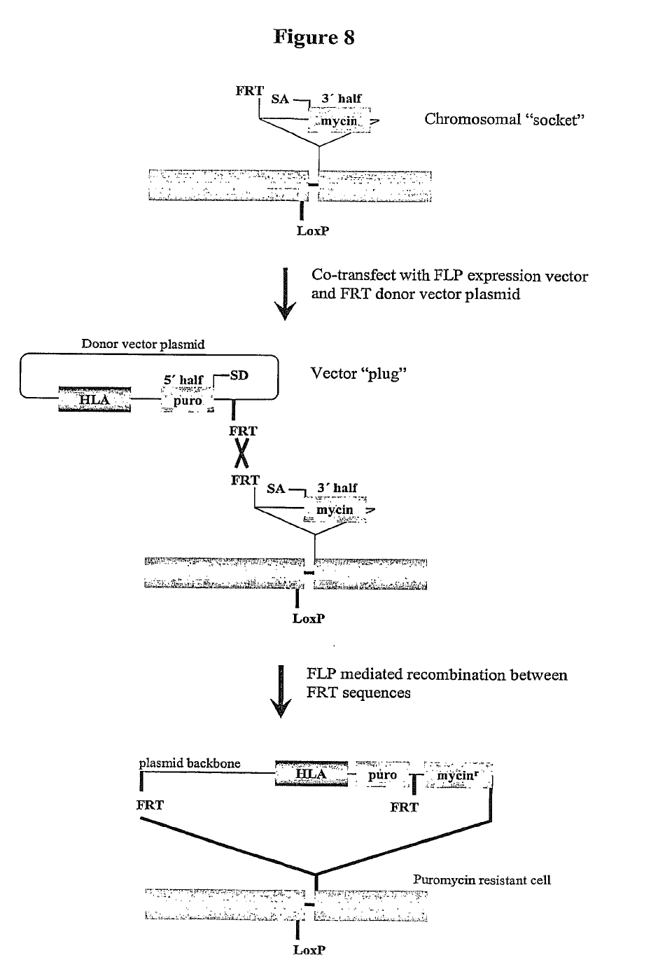

Introduction of defined MLA genes into the gene modified cell lines is accomplished using a “plug and socket” site specific recombination strategy. In this approach, an inactive “socket” gene fragment is retained in the targeted chromosome (FIG. 8). In FIG. 8, the chromosomal socket is the 3′ portion of the puromycin gene and the 5′ portion of the puromycin gene is the plug. Other drug selectable markers, visually screenable markers and FACS markers described above could be engineered to work as a plug and socket pair. Site specific recombination between the plug and socket pair reconstitutes the functioning puromycin acetyl transferase gene conferring cellular growth in the presence of puromycin. Genes to be introduced at the “socket site” are present on the plug vector. In this example, cotransfection of the plug vector with the expression cassette for the FLP recombinase generates puromycin resistant cell lines with the desired HLA alleles expressed.

### Example 6

### Modification of Isolated Chromosomes and Chromatin by Recombinase Treated Targeting Vectors or Oligonucleotides to Engineer Cells with Defined HLA or ABO

The DNA from cell free chromosomes and chromatin, can be genetically modified enzymatically with targeting vectors or oligonucleotides, using purified recombinases or purified DNA repair proteins. The targeting DNAs may have tens of kilobasepairs to oligonucleotides of at least 50 basepairs of homology to the chromosomal target. Recombinase catalyzed recombination intermediates formed between target chromosomes and vector DNA can be enzymatically resolved in cell free extracts with other purified recombination or DNA repair proteins to produce genetically modified chromosomes. These modified chromosomes can be reintroduced into cells or for formation of nuclei in vitro prior to introduction into cells. Recombinase treated vector or oligonucleotides can also be directly introduced into isolated nuclei by microinjection or by diffusion into permeabilized nuclei to allow in situ formation of recombination intermediates that can be resolved in vitro, on nuclear transfer into intact cells, or on fusion with recipient cells.

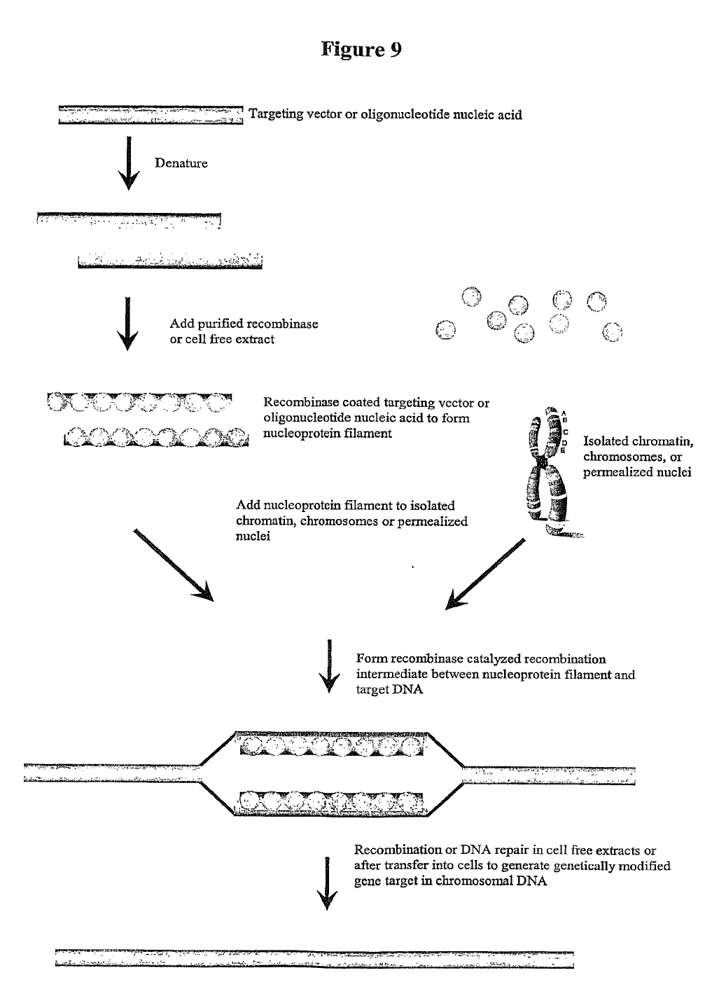

In this approach, enzymatically active nucleoprotein filaments are first formed between targeting vector, or oligonucleotides, and recombinase proteins. Recombinase proteins are cellular proteins that catalyze the formation of heteroduplex recombination intermediates intracellularly and can form similar intermediates in cell free systems. Well studied, prototype recombinases are the RecA protein from E. coli and Rad51 protein from eukaryotic organisms. Recombinase proteins cooperatively bind single stranded DNA and actively catalyze the search for homologous DNA sequences on other target chromosomal DNAs. Heteroduplex structures may also be formed and resolved using cell free extracts from cells with recombinogenic phenotypes. In a second step, heteroduplex intermediates may be resolved in cell free extracts by treatment with purified recombination and DNA repair proteins to recombine the donor targeting vector DNA or oligonucleotide into the target chromosomal DNA (FIG. 9). This may also be accomplished using cell free extracts from normal cells or extracts from cells with a recombinogenic phenotype. Finally, the nuclear membrane is reformed around modified chromosomes and the remaining unmodified cellular chromosomal complement for introduction into recipient cells or oocytes.

Construction of ABO alpha 1-3-N-acetylgactosaminyltransferase/alpha-3D-galactosyltransferase gene targeting probe

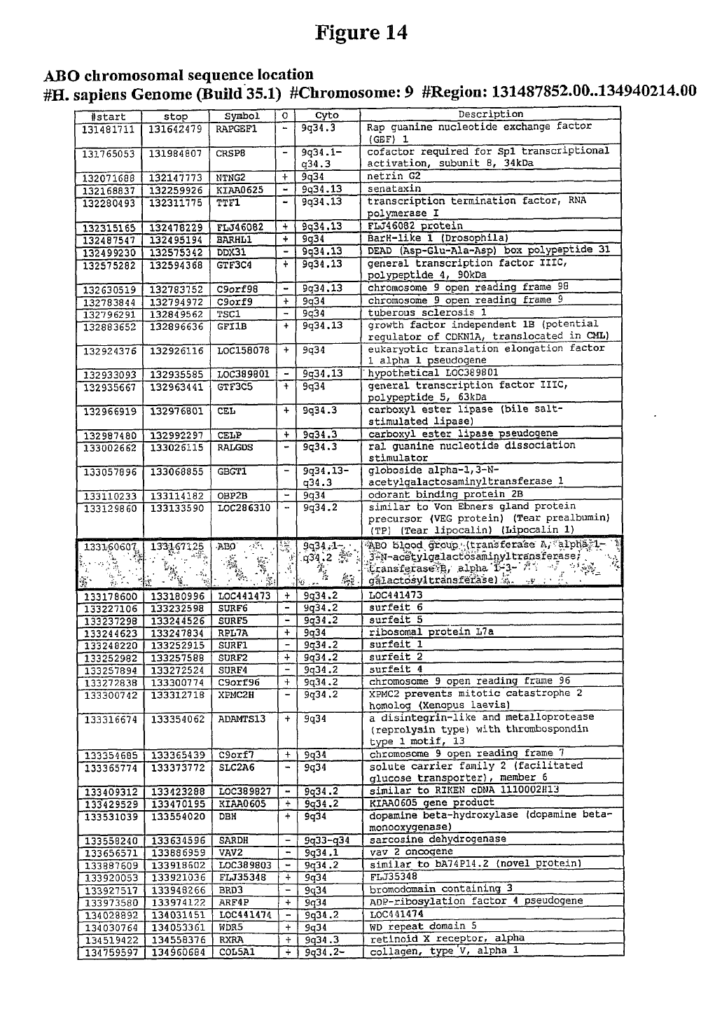

This targeting strategy is designed to inactivate the type A (alpha 1-3-N-acetylgactosaminyltransferase) or type B (alpha-3-D-galactosyltransferase) allele of the blood group ABO transferase gene to generate a type O phenotype. The human ABO genes consist of at least 7 exons, and the coding sequence in the 7 coding exons spans over 18 kb of genomic DNA. The exons range in size from 28 to 688 bp, with most of the coding sequence lying in exon 7 (FIG. 14).

The gene targeting probe with an O type allele, a deletion of guanine at nucleotide 258 of the coding sequence, is amplified directly from DNA from an O type tissue sample. The PCR oligonucleotides are located approxlmately 250 haee pairs 5′ and 3′ to the nucleotide 258 mutation. Deletion of the guanine residue at 258 inactivates a BstEII restriction endonuclease site and activates a KpnI restriction endonuclease site enabling a convenient screen for gain of a KpnI restriction site in the genomic DNA as a consequence of a successful gene targeting event. Genomic DNA from tissue samples is prepared using standard methods and may be performed using kits such as those provided by Qiagen. PCR reactions contain genomic DNA, PCR oligonucleotides, Taq polymerase, buffer and deoxyribonucleotides as described by the manufacturer. The sequence of the 5′ PCR oligonucleotide is, for example, 5′-GGGTTTGTTCCTATCTCTTTG-3′ SEQ ID NO: 1) and the sequence of the 3′ PCR oligonucleotide is, for example, 5′-GACCTGGCGAGCCCACGAG-3′ (SEQ ID NO.: 2). The 500 basepair PCR product is gel purified and used for coating by the 30 RecA or Rad51 recombinase.

Forming Recombinase Coated Nucleoprotein Filaments

Circular DNA targeting vectors are first linearized by treatment with restriction endonucleases, or alternatively linear DNA molecules are produced by PCR from genomic DNA or vector DNA. All DNA targeting vectors and traditional DNA constructs are removed from vector sequences by agarose gel electrophoresis and purified with Elutip-D columns (Schleicher & Schuell, Keene, N.H.). For RecA protein coating of DNA, linear, double-stranded DNA (200 ng) is heat denatured at 98° C. for 5 minutes, cooled on ice for 1 minute and added to protein coating mix containing Tris-acetate buffer, 2 mM magnesium acetate and 2.4 mM ATPγS. RecA protein (8.4 μg) is immediately added, the reaction incubated at 37° C. for 15 minutes, and magnesium acetate concentration increased to a final concentration of 11 mM. The RecA protein coating of DNA is monitored by agarose gel electrophoresis with uncoated double-stranded DNA as control. The electrophoretic mobility of RecA-DNA is significantly retarded as compared with non-coated double stranded DNA.

Isolation of Cell Free Chromosomes and Chromatin

Donor fibroblasts are exposed to conditions that remove the plasma membrane, resulting in the isolation of nuclei. These nuclei, in turn, are exposed to cell extracts that result in nuclear envelope dissolution and chromatin condensation. Dermal fibroblasts are cultured in DMEM with 10% fetal calf serum until the cells reach confluence. Approximately 1×10⁶ cells are then harvested by trypsinization, the trypsin is inactivated, and the cells are suspended in 50 mL of phosphate buffered saline (PBS), pelleted by centrifuging the cells at 500×g for 10 minutes at 4° C., the PBS is discarded, and the cells are resuspended in 50 times the volume of the pellet in ice-cold PBS, and centrifuged as above. Following this centrifugation, the supernatant is discarded and the pellet is resuspended in 50 times the volume of the pellet of hypotonic buffer (10 mM HEPES, pH 7.5, 2 mM MgCl₂, 25 mM KCl, 1 mM DTT, 10 μM aprotinin, 10 μM leupeptin, 10 μM pepstatin A, 10 μM soybean trypsin inhibitor, and 100 μM PMSF) and again centrifuged at 500×g for 10 min at 4° C. The supernatant is discarded and 20 times the volume of the pellet of hypotonic buffer is added and the cells are carefully resuspended and incubated on ice for an hour. The cells are then physically lysed. Briefly, 5 ml of the cell suspension is placed in a glass bounce homogenizer and homogenized with 20 strokes. Cell lysis is monitored microscopically to observe the point where isolated and yet undamaged nuclei result. Sucrose is added to make a final concentration of 250 mM sucrose (⅛ volume of 2 M stock solution in hypotonic buffer). The solution is carefully mixed by gentle inversion and then centrifuged at 400×g at 4° C. for 30 minutes. The supernatant is discarded and the nuclei are then gently resuspended in 20 volumes of nuclear buffer (10 mM HEPES, pH 7.5, 2 mM MgCl₂, 250 mM sucrose, 25 mM KCl, 1 mM DTT, 10 μM aprotinin, 10 μM leupeptin, 10 μM pepstatin A, 10 μM soybean trypsin inhibitor, and 100 μM PMSF). The nuclei are re-centrifuged as above and resuspended in 2 times the volume of the pellet in nuclear buffer. The resulting nuclei may then be used directly for gene modifications, nuclear remodeling, or cryopreserved for future use.

Extract for Nuclear Envelope Breakdown and Chromatin Condensation

The condensation extract, when added to the isolated differentiated cell nuclei, will result in nuclear envelope breakdown and the condensation of chromatin. A separate extract is used for nuclear envelope reconstitution after cell free homologous recombination reactions have modified target chromosomes. Extract for nuclear envelope breakdown and chromatin condensation, and for nuclear envelope reconstitution may be prepared from any proficient mammalian cell line. However, extracts from the human embryonal carcinoma cell line NTera-2 can be potentially used for the condensation extract and for nuclear envelope reconstitution extract as well as for remodeling differentiated chromatin to an undifferentiated state, thus enhancing production of genetically modified human ES cells starting from differentiated human dermal cells. NTera-2 cl. D1 cells are easily obtained from sources such as the American Type Culture Collection (CRL-1973) and are grown at 37° C. in monolayer culture in DMEM with 4 mM L-glutamine, 1.5 g/L sodium bicarbonate and 4.5 g/L glucose, 10% fetal bovine serum (complete medium). While in a log growth state, the cells are plated at 5×10⁶ cells per sq cm tissue culture flask in 200 mL of complete medium. Methods of obtaining extracts capable of inducing nuclear envelope breakdown and chromosome condensation are well known in the art (Collas et al., J. Cell Biol. 147:1167-1180, (1999)). Briefly, NTera-2 cells in log growth as described above are synchronized in mitosis by incubation in 1 μg/ml nocodazole for 20 hours. The cells that are in the mitotic phase of the cell cycle are detached by mitotic shakeoff. The harvested detached cells are centrifuged at 500×g for 10 minutes at 4° C. Cells are resuspended in 50 ml of cold PBS, and centrifuged at 500×g for an additional 10 min at 4° C. This PBS washing step is repeated once more. The cell pellet is then resuspended in 20 volumes of ice-cold cell lysis buffer (20 mM HEPES, pH 8.2, 5 mM MgCl₂, 10 mM EDTA, 1 mM DTT, 10 μM aprotinin, 10 μM leupeptin, 10 μM pepstatin A, 10 μM soybean trypsin inhibitor, 100 μM PMSF, and 20 μg/ml cytochalasin B, and the cells are centrifuged at 800×g for 10 minutes at 4° C. The supernatant is discarded, and the cell pellet is carefully resuspended in one volume of cell lysis buffer. The cells are placed on ice for one hour then lysed with a Dounce homogenizer. Progress is monitored by microscopic analysis until over 90% of cells and cell nuclei are lysed. The resulting lysate is centrifuged at 15,000×g for 15 minutes at 4° C., the tubes are then removed and immediately placed on ice. The supernatant is gently removed using a small caliber pipette tip, and the supernatant from several tubes is pooled on ice. If not used immediately, the extracts are immediately flash-frozen on liquid nitrogen and stored at −80° C. until use. The cell extract is then placed in an ultracentrifuge tube and centrifuged at 200,000×g for three hours at 4° C. to sediment nuclear membrane vesicles. The supernatant is then gently removed and placed in a tube on ice and used immediately to prepare condensed chromatin or cryopreserved as described above.

Extract for Nuclear Envelope Reconstitution

Nuclear envelope reconstitution extract is prepared using NTera-2 cl. D1 cells obtained from sources such as the American Type Culture Collection. While in a log growth state, the cells are plated at 5×10⁶ cells per sq. cm tissue culture flask in 200 mL of complete medium. Extracts from cells in the prometaphase are prepared as is known in the art (Burke & Gerace, Cell 44: 639-652, (1986)). Briefly, after two days and while still in a log growth state, the medium is replaced with 100 mL of complete medium containing 2 mM thymidine (which sequesters the cells in S phase). After 11 hours, the cells are rinsed once with 25 mL of complete medium, then incubated with 75 mL of complete medium for four hours, at which point nocodazole is added to a final concentration of 600 ng/mL from 10,000× stock solution in DMSO. After one hour, loosely attached cells are removed by mitotic shakeoff (Tobey et al., J. Cell Physiol. 70:63-68, (1967)). This first collection of removed cells is discarded, the medium is replaced with 50 mL of complete medium also containing 600 ng/mL of nocodazole. Prometaphase cells are then collected by shakeoff 2-2.5 hours later. The collected cells are then incubated at 37° C. for 45 minutes in 20 mL of complete medium containing 600 ng/mL nocodazole and 20 μM cytochalasin B. Following this incubation, the cells are washed twice with ice-cold Dulbecco's PBS, then once in KHM (78 mM KCl, 50 mM Hepes-KOH [pH 7.0], 4.0 mM MgCl₂, 10 mM EGTA, 8.37 mM CaCl₂, 1 mM DTT, 20 μM cytochlasin B). The cells are then centrifuged at 1000×g for five minutes, the supernatant discarded, and the cells resuspended in the original volume of KHM. The cells are then homogenized in a dounce homogenizer on ice with about 25 strokes and progress determined by microscopic observation. When at least 95% of the cells are homogenized extracts held on ice for use in envelope reassembly or cryopreserved as is well known in the art.

Treatment for Nuclear Membrane Breakdown and Chromosomal Condensation

For nuclear membrane breakdown and chromosomal condensation, isolated nuclei are treated with the extract described above. If beginning with a frozen aliquot of condensation extract, the frozen extract is thawed on ice. Then an ATP-generating system is added to the extract such that the final concentrations are 1 mM ATP, 10 mM creatine phosphate, and 25 μg/ml creatine kinase. The nuclei isolated from the differentiated cells as described above are then added to the extract at 2,000 nuclei per 10 μl of extract, mixed gently, and then incubated in a 37° C. water bath. The tube is removed occasionally to gently resuspend the cells by tapping on the tube. Extracts and cell sources vary in times for nuclear envelope breakdown and chromosome condensation. Therefore the progress is monitored by periodically monitoring the samples microscopically. When the majority of cells have lost their nuclear envelope and there is evidence of the beginning of chromosome condensation, the extract containing the condensing chromosome masses is placed in a centrifuge tube with an equal volume of 1 M sucrose solution in nuclear buffer. The chromatin masses are sedimented by centrifugation at 1,000×g for 20 minutes at 4° C.

Forming Heteroduplex Recombination Intermediates Between Preformed Recombinase Coated Nucleoprotein Targeting Vectors and Oligonucleotides and Cell Free Chromosomes and Chromatin

Formation of targeting vector/chromosome heteroduplexes is performed by adding approximately 1-3 μg of double-stranded chromosomal DNA or chromatin masses to the RecA coated nucleoprotein filaments described above, and incubated at 37° C. for 20 minutes. If the nucleoprotein heteroduplex structures are to be deproteinized prior to additional in vitro recombination steps, they are treated by with the addition of SDS to a final concentration of 1.2%, or by addition of proteinase K to 10 mg/ml with incubation for 15 to 20 minutes at 37° C., followed by addition of SDS to a final concentration of 0.5 to 1.2% (wt/vol). Residual SDS is removed prior to subsequent steps by microdialysis against 100 to 1000 volumes of protein coating mix.

Resolving Recombination Intermediates with Cell Free Extracts

Cell free extracts may be prepared from normal fibroblast or hES cell lines, or may be prepared from cells demonstrated to have recombinogenic phenotypes. Cell lines exhibiting high levels of recombination in vivo are the chicken pre-B cell line DT40 and the human lymphoid DG75 cell line. Preparation of cell free extracts is performed at 4° C. About 10⁸ actively growing cells are harvested from either dishes or suspension cultures. The cells are washed three times with phosphate-buffered saline (PBS; 140 mM NaCl, 3 mM KCl, 8 mM NaH₂PO₄, 1 mM K₂HPO₄, 1 mM MgCl₂, 1 mM CaCl₂), resuspended in 2 to 3 ml of hypotonic buffer A (10 mM Tris hydrochloride [pH 7.4], 10 mM MgCl₂, 10 mM KCl, 1 mM dithiothreitol), and kept on ice for 10 to 15 minutes. Phenylmethylsulfonyl fluoride is added to a concentration of 1 mM, and the cells are broken by 5 to 10 strokes in a Bounce homogenizer, pestle B. The released nuclei are centrifuged at 2,600 rpm in a Beckman TJ-6 centrifuge for 8 min. The supernatant is removed carefully and stored in 10% glycerol-100 mM NaCl at −70° C. (cytoplasmic fraction). The nuclei are resuspended in 2 ml of buffer A containing 350 mM NaCl, and the following proteinase inhibitors are added: pepstatin to a concentration of 0.25 μg/ml, leupeptin to a concentration of 0.1 μg/ml, aprotinin to a concentration of 0.1 μg/ml, and phenylmethylsulfonyl fluoride to a concentration of 1 mM (all from Sigma Chemicals). After 1 h of incubation at 0° C., the extracted nuclei are centrifuged at 70,000 rpm in a Beckman TL-100/3 rotor at 2° C. The clear supernatant is adjusted to 10% glycerol, 10 mM β-mercaptoethanol and frozen immediately in liquid nitrogen prior to storage at −70° C. (fraction 1).

To resolve recombination intermediates in vitro, chromosomal heteroduplex intermediates are incubated with 3 to 5 μg of extract protein in a reaction mixture containing 60 mM NaCl, 2 mM 3-mercaptoethanol, 2 mM KCl, 12 mM Tris hydrochloride (pH 7.4), 1 mM ATP, 0.1 mM each deoxyribonucleoside triphosphate (dNTP), 2.5 mM creatine phosphate, 12 mM MgCl₂, 0.1 mM spermidine, 2 glycerol, and 0.2 mM dithiothreitol. After 30 minutes at 37° C., the reaction is stopped by the addition of EDTA to a concentration of 25 μM, sodium dodecyl sulfate (SDS) to a concentration of 0.5%, and 20 μg of proteinase K and incubated for 1 hour at 37° C. SDS is removed prior to subsequent steps by microdialysis. An equal volume of 1 M sucrose is added to the treated chromatin masses and sedimented by centrifugation at 1,000×g for 20 minutes at 4° C.

Reforming Nuclear Envelopes Around Recombinant Chromosomes and Chromatin

The supernatant is discarded, and the chromatin masses are gently resuspended in nuclear remodeling extract described above. The sample is then incubated in a water bath at 33° C. for up to two hours and periodically monitored microscopically for the formation of remodeled nuclear envelopes around the condensed and remodeled chromatin as described (Burke & Gerace, Cell 44:639-652, (1986). Once a large percentage of chromatin has been encapsulated in nuclear envelopes, the remodeled nuclei may be used for cellular reconstitution using any of the techniques described in the present invention.

Detection of Cells Containing Genetically Modified Chromosomes

Reconstituted cells are grown for 7 to 14 days and screened for recombinants using PCR and Southern hybridization.

### Example 7

### Modification of Chromosomes and Chromatin in Isolated Nuclei with Targeting Vectors or Oligonucleotides to Engineer Cells with Defined HLA or ABO

Chromosomes and chromatin may be genetically modified in isolated nuclei from cells. In this approach, intact nuclei are isolated from growing cells, and reversibly permeabilized to allow diffusion of nucleoprotein targeting vectors and oligonucleotides into the nucleus interior. Heteroduplex intermediates formed between nucleoprotein targeting vectors and oligonucleotides and chromosomal DNA may be resolved by treatment with recombination proficient cell extracts, purified recombination and DNA repair proteins, or by cellular reconstitution with the nuclei into recombination proficient cells.

Isolation and Permeabilization of Nuclei

Preparation of Synchronous Populations of Nuclei Cell culture and synchronization are carried out as previously described ((Leno et al., Cell 69:151-158 (1992)). Nuclei are prepared as described except that all incubations are carried out in HE buffer (50 mM Hepes-KOH, pH 7.4, 50 mM KCl, 5 mM MgCl2, 1 mM EGTA, 1 mM DTT, 1 μg/ml aprotinin, pepstatin, leupeptin, chymostatin).

Nuclear Membrane Permeablization Streptolysin O (SLO)-prepared nuclei (Lena et al., Cell 69:151-158 (1992)) are incubated with 20 μg/ml lysolecithin (Sigma Immunochemicals) and 10/μg/ml cytochalasin B in HE at a concentration of −1.5×10⁴ nuclei/ml for 10 min at 23° C. with occasional gentle mixing. Reactions are stopped by the addition of 1% nuclease free BSA (Sigma Immunochemicals). Nuclei are gently pelleted by centrifugation in a RC5B rotor (Sorvall Instruments, Newton, Conn.) at 500 rpm for 5 min and then washed three times by dilution in 1 ml HE. Pelleted nuclei are recovered in a small volume of buffer and resuspended to −1×10⁴ nuclei/μl.

Forming Heteroduplex Recombination Intermediates Between Preformed Recombinase Coated Nucleoprotein Targeting Vectors and Oligonucleotides and Cell Free Chromosomes and Chromatin

Formation of targeting vector/chromosome heteroduplexes is performed by adding approximately 1×10⁵ to 1×10⁶ permeabilized nuclei to the RecA coated nucleoprotein filaments described above, and incubated at 37 for 20 minutes.

Resolving Recombination Intermediates with Cell Free Extracts

Cell free extracts may be prepared from normal fibroblast or hES cell lines, or may be prepared from cells demonstrated to have recombinogenic phenotypes. Cell lines exhibiting high levels of recombination in vivo are the chicken pre-B cell line DT40 and the human lymphoid DG75 cell line. Preparation of cell free extracts are performed at 4° C. About 10⁸ actively growing cells are harvested from either dishes or suspension cultures. The cells are washed three times with phosphate-buffered saline (PBS; 140 mM NaCl, 3 mM KCl, 8 mM NaH₂PO₄, 1 mM K₂HPO₄, 1 mM MgCl₂, 1 mM CaCl₂), resuspended in 2 to 3 ml of hypotonic buffer A (10 mM Trio hydrochloride [pH 7.4], 10 mM MgCl₂, 10 mM KCl, 1 mM dithiothreitol), and kept on ice for 10 to 15 minutes. Phenylmethylsulfonyl fluoride is added to 1 mM, and the cells are broken by 5 to 10 strokes in a Dounce homogenizer, pestle B. The released nuclei are centrifuged at 2,600 rpm in a Beckman TJ-6 centrifuge for 8 min. The supernatant is removed carefully and stored in 10% glycerol-100 mM NaCl at −70° C. (cytoplasmic fraction). The nuclei are resuspended in 2 ml of buffer A containing 350 mM NaCl, and the following proteinase inhibitors are added: pepstatin to 0.25 μg/ml, leupeptin to 0.1 μg/ml, aprotinin to 0.1 μg/ml, and phenylmethylsulfonyl fluoride to 1 mM (all from Sigma Chemicals). After 1 h of incubation at 0° C., the extracted nuclei are centrifuged at 70,000 rpm in a Beckman TL-100/3 rotor at 2° C. The clear supernatant is adjusted to 10% glycerol, 10 mM β-mercaptoethanol and frozen immediately in liquid nitrogen prior to storage at −70° C. (fraction 1).

To resolve recombination intermediates in permeabilized nuclei, nuclei containing chromosomal heteroduplex intermediates are incubated with 3 to 5 μg of extract protein in a reaction mixture containing 60 mM NaCl, 2 mM 3-mercaptoethanol, 2 mM KCl, 12 mM Tris hydrochloride (pH 7.4), 1 mM ATP, 0.1 mM each deoxyribonucleoside triphosphate (dNTP), 2.5 mM creatine phosphate, 12 mM MgCI₂, 0.1 mM spermidine, 2% glycerol, and 0.2 mM dithiothreitol. After 30 minutes at 37° C., the reaction is stopped.

Nuclear Envelope Repair

Preparation and Fractionation of Nuclear Repair Extract

Low-speed Xenopus egg extracts (LSS) 1 are prepared essentially according to the procedure described by Blow and Lackey Cell 21; 47:577-87 (1586)). Extraction buffer (50 mM Hepes-KOH, pH 7.4, 50 mM KCl, 5 mM MgCI₂) is thawed and supplemented with 1 mM DTT, 1 μg/ml leupeptin, pepstatin A, chymostatin, aprotinin, and 10 μg/ml cytochalasin E (Sigma immunochemicals, S. Louis, Mo.) immediately before use. Extracts are supplemented with 2% glycerol and snap-frozen as 10-20 μl beads in liquid nitrogen or subjected to further fractionation. High speed supernatant (HSS) and membrane fractious are prepared from low-speed egg extract as described (Sheehan et al., J Cell Biol. 106:1-12 (1988)). Membranous material, isolated by centrifugation of 1-2 ml of low-speed extract, is washed at least two times by dilution in 5 ml extraction buffer. Diluted membranes are centrifuged for 10 minutes at 10 k rpm in an SW50 rotor (SW50; Beckman Instruments, Inc., Palo Alto, Calif.) to yield vesicle fraction 1. The supernatant is then centrifuged for a further 30 min at 30 k rpm to yield vesicle fraction 2. Washed membranes are supplemented with 5% glycerol and snap-frozen in 5 μl beads in liquid nitrogen. Vesicle fractions 1 and 2 are mixed in equal proportions before use in nuclear membrane repair reactions.

Treatment for Nuclear Envelope Repair

Lysolecithin-permeabilized nuclei are repaired by incubation with membrane components prepared from Xenopus egg extracts. Nuclei at a concentration of approximately 5000/μl are mixed with an equal volume of pooled vesicular fractions 1 and 2 and supplemented with 1 mM GTP and ATP. 10-20-μl reactions are incubated at 23° C. for up to 90 min with occasional gentle mixing. Aliquots are taken at intervals and assayed for nuclear permeability.

Once a large percentage of chromatin is encapsulated in nuclear envelopes, the remodeled nuclei may be used for cellular reconstitution using any of the techniques described in the present invention.

Detection of Cells Containing Genetically Modified Chromosomes

Reconstituted cells are grown for 7 to 14 days and screened for recombinants using PCR and Southern hybridization.

### Example 8

### Modification of Isolated Chromosomes, Chromatin, and Nuclei Using Cell Free Extracts to Engineer Cells with Defined HLA or ABO

In this approach, targeting vectors or oligonucleotides and the target chromosomal DNA are directly treated with recombination proficient cell free extracts from cells with recombinogenic phenotypes such as the chicken pre-2 cell line DT40 and the human lymphoid cell line DG75. These cell free extracts may be used on isolated chromosome and chromatin or on isolated permeabilized nuclei. Essentially, targeting vector/oligonucleotides are incubated with isolated chromosomes, chromatin, or nuclei and cell free recombination extract. The nuclear envelope is reconstituted around recombinant chromosomes or chromatin, or the nuclear envelope of recombinant, permeabilized, nuclei are repaired prior to cell reconstitution with the reconstituted or repaired nuclei.

Preparation of Cell Free Extracts

Cell free extracts from DT40 or DG75 cell are prepared as described above.

Preparation of Chromosomes, Chromatin, or Nuclei

Isolated chromosomes, chromatin, and permeabilized nuclei from fibroblasts, hES cell lines, or germ cell lines are as described above.

Recombination between targeting vectors and oligonucleotides, and cell free chromosomes and chromatin using cell free extracts from recombinogenic cells.

Circular DNA targeting vectors are first linearized by treatment with restriction endonucleases, or alternatively linear DNA molecules are produced by PCR from genomic DNA or vector DNA. All DNA targeting vectors and traditional DNA constructs are removed from vector sequences by agarose gel electrophoresis and purified with Elutip-D columns (Schleicher & Schuell, Keene, N.H.). Double-stranded DNA (200 ng) is heat denatured at 98° C. for 5 minutes, cooled on ice for 1 minute and added to approximately 1-3 μg of double-stranded chromosomal DNA or chromatin masses, or approximately 1×10⁵ to 1×10⁶ permeabilized nuclei, and 3 to 5 μg of extract protein in a reaction mixture containing 60 mM NaCl, 2 mM 3-mercaptoethanol, 2 mM KCl, 12 mM Tris hydrochloride (pH 7.4), 1 mM ATP, 0.1 mM each deoxyribonucleoside triphosphate (dNTP), 2.5 mM creatine phosphate, 12 mM MgCI₂, 0.1 mM spermidine, 2% glycerol, and 0.2 mM dithiothreitol. The reaction mixtures are incubated at 37° C. for at least 30 minutes are processed as describe above prior to reconstituting cellular envelopes or repairing permeabilized nuclei.

Reforming Nuclear Envelopes Around Recombinant Chromosomes and Chromatin

Nuclear envelopes are reconstituted around recombinant chromosomes and chromatin and reconstituted nuclei used for cellular reconstitution as describe above.

Nuclear Envelope Repair

Recombinant, permeabilized nuclei are repaired and repaired recombinant nuclei used for cellular reconstitution as described above.

Detection of Cells Containing Genetically Modified Chromosomes

Reconstituted cells are grown for 7 to 14 days and screened for recombinants using PCR and Southern hybridization.

### Example 9

### Modification of Chromosomes and Chromatin in Intact Cells with Recombinase Treated Targeting Vectors or Oligonucleotides to Engineer Cells with Defined HLA or ABO

In this approach, double stranded targeting vectors, targeting DNA fragments, or oligonucleotides are coated with bacterial or eukaryotic recombinase and introduced into mammalian cells or oocytes. The activated nucleoprotein filament forms heteroduplex recombination intermediates with the chromosomal target DNA that is subsequently resolved to a homologous recombinant structure by the cellular homologous recombination or DNA repair pathways. While the most direct delivery of nucleoprotein filaments is by direct nuclear/pronuclear microinjection, other delivery technologies can be used including electroporation, chemical transfection, and single cell electroporation.

To form human Rad51 nucleoprotein filaments, linear, double-stranded DNA (200 ng) is heat denatured at 98° C. for 5 minutes, cooled on ice for 1 minute and added to a protein coating mix containing 25 mM Tris acetate (pH 7.5), 100 μg/ml BSA, 1 nM DTT, 20 mM KCl (added with the protein stock), 1 mM ATP and 5 mM CaCl₂, or AMP-PNP and 5 mM MgCl₂. hRad51 protein (1 μM) is immediately added and the reaction incubated at for 10 minutes at 37° C. The hRad51 protein coating of the DNA is monitored by agarose gel electrophoresis with uncoated double-stranded DNA as control. The electrophoretic mobility of hRad51-DNA nucleoprotein filament is significantly retarded as compared with non-coated double stranded DNA. hRad51-DNA nucleoprotein filaments are diluted to a concentration of 5 ng/μl and used for nuclear microinjection of human fibroblasts or somatic cells, or used for pronuclear microinjection of activated oocytes created by somatic cell nuclear transfer or in vitro fertilization.

Detection of Cells Containing Genetically Modified Chromosomes

Injected cells or oocytes axe grown for 7 to 14 days and screened for recombinants using PCR and Southern hybridization.

1-11. (canceled)

12. A bank of totipotent, nearly totipotent and/or pluripotent stem cells, comprising a library of human or non-human animal stem cells, each of which cells is hemizygous or homozygous for at least one MHC allele present in a human or non-human animal population, wherein said bank of stem cells comprise stem cells that are hemizygous or homozygous for different sets of MHC alleles relative to the other members in the bank of stem cells, and wherein gene targeting and/or loss of heterozygosity is used to generate the hemizygous or homozygous MHC allele.

13. A method of generating a stem cell hemizygous or homozygous for at least one MHC allele, comprising deleting one or two of the two MHC alleles in a stem cell by gene targeting.

14. The method of claim 13 further comprising using loss of heterozygosity to generate a stem cell homozygous for at least one MHC allele from said stem cell that is hemizygous for at least one MHC allele, thereby generating a stem cell homozygous for at least one MHC allele.

15. The method according to claim 13, further comprising destabilizing or inactivating p53 by expressing the human papiloma virus E6 protein or adenovirus E1B gene.

16-22. (canceled)

23. An isolated parthenogenic ES cell or isolated ES cell derived by parthenogenesis which ES cell is homozygous for one or more histocompatibility antigens.

24. The stem cell according to claim 23, wherein said stem cell is homozygous for at least one MHC allele present in a human or non-human animal population.

25. The stem cell according to claim 24, wherein said at least one MHC allele is generated by gene targeting to arrive at a hemizygous allele and then by loss of heterozygosity to arrive at a homozygous allele.

26. The stem cell according to claim 23, further comprising one or more drug selectable markers.

27. The stem cell according to claim 23, further comprising nucleic acid sequences encoding recognition sequences for the Cre/LoxP or the FLP/FRT recombinases.

28. The stem cell according to claim 23, further comprising nucleic acid sequences encoding the recognition sequence for the I-SceI endonuclease.

29. The stem cell according to claim 26, wherein the drug selectable marker is used to positively select cells that are hemizygous or homozygous for at least one MHC allele.

30. The stem cell according to claim 26, wherein the drug selectable marker is used to negatively select cells that are hemizygous or homozygous for at least one MHC allele.

31. The stem cell according to claim 23, wherein said cell is O-negative.

32. The stem cell according to claim 31, wherein said cell is generated from a female.

33. The stem cell of claim 23 which is contained in a library of isolated parthenogenic ES cell or isolated ES cell derived by parthenogenesis, each of which cells is hemizygous or homozygous for at least one MHC allele present in a human or non-human animal population, wherein said bank of stem cells comprise stem cells that are hemizygous or homozygous for different sets of MHC alleles relative to the other members in the bank of stem cells, and wherein gene targeting and/or loss of heterozygosity is used to generate the hemizygous or homozygous MHC allele.
